(19) United States
(12) Patent Application Publication
(10) Pub. No.: US 2003/0027223 A1
(43) Pub. Date: Feb. 6, 2003

(54) Specimen-linked G protein coupled receptor database

(76) Inventor: Patrick J. Muraca, Pittsfield, MA (US)

Correspondence Address: PALMER & DODGE, LLP, PAULA CAMPBELL EVANS, 111 HUNTINGTON AVENUE, BOSTON, MA, 02199, US

(21) Appl. No.: 10/184,694

(22) Filed: Jun. 28, 2002

Related U.S. Application Data
(60) Provisional application No. 60/302,316, filed on Jun. 29, 2001.

(51) Int. Cl. G01N 33/567; G06F 19/00; G01N 33/48; G01N 33/50

(52) U.S. Cl. 435/7.21; 702/19

(57) ABSTRACT

The invention relates to a method and system for identifying and evaluating the physiological responses of an organism to a condition, such as a disease or other pathological condition, a drug or agent, an environmental condition, and the like, by evaluating the expression of one or more GPCR pathway biomolecules in tissue microarrays from a plurality of patients. In one aspect, a tissue information system is provided comprising a specimen-linked database and an information management system for accessing, organizing, and displaying tissue information obtained from tissue microarrays. Preferably, the system is used to model and validate GPCR pathways affected during one or more physiological responses to a condition.

### RELATED APPLICATIONS

This application claims priority under 35 U.S.C.§119(e) to U.S. Serial No. 60/302,316, filed Jun. 29, 2001. The entire teachings of the above application are incorporated herein by reference.

### FIELD OF THE INVENTION

The invention relates to a database which links information relating to the expression of G protein coupled receptors (GPCR) in a plurality of tissue microarrays with the characteristics of patients from whom these samples derive.

### BACKGROUND

G-protein coupled receptors (GPCR) are a large group of receptors which transduce extracellular signals. The structure of these highly-conserved receptors consists of seven hydrophobic transmembrane regions, an extracellular N-terminus, and a cytoplasmic C-terminus. The N-terminus interacts with ligands, and the C-terminus interacts with intracellular G proteins to activate second messengers such as cyclic AMP (cAMP), phospholipase C, inositol triphosphate, or ion channel proteins (see, e.g., Baldwin, Curr. Opin. Cell Biol. 6: 180-190 (1994). The amino-terminus of the GPCR is extracellular, of variable length and often glycosylated, while the carboxy-terminus is cytoplasmic and generally phosphorylated. GPCRs respond to a diverse array of ligands including lipid analogs, amino acids and their derivatives, peptides, cytokines, and as well to stimuli such as light, taste, and odor. GPCRs function in physiological processes including vision (e.g., rhodopsins), smell (e.g., olfactory GPCR receptors), neurotransmission (e.g., muscarinic acetylcholine, dopamine, and adrenergic receptors), and hormonal responses (e.g., luteinizing hormone and thyroid-stimulating hormone receptors).

GPCRs include receptors for biogenic amines such as dopamine, epinephrine, histamine, glutamate (metabotropic effect), acetylcholine (muscarinic effect), and serotonin; for lipid mediators of inflammation such as prostaglandins, platelet activating factor, and leukotrienes; for peptide hotmones such as calcitonin, C5a anaphylatoxin, follicle stimulating hormone, gonadotropin releasing hormone, neurokinin, oxytocin, and thrombin; and for sensory signal mediators such as retinal photopigments and olfactory stimulatory molecules.

Mutations in genes encoding GPCRs have been associated with diseases in humans (see, e.g., Coughlin, Curr. Opin. Cell Biol. 6:191-197, 1994). Both loss-of-function and gain-of-function have been reported. For example, both loss-of-function and gain-of-function mutations in the rhodopsin gene have been associated with retinitis pigmentosa. Gain-of-function mutations in the thyrotropin receptor have likewise been associated with hyperfunctioning thyroid adenomas (Parma, J. et al. Nature 365: 649-651, (1993)). There has been a suggestion that gain-of-function mutations in GPCRs can behave like proto-oncogenes. See, e.g., Parma et al., supra.

In addition, GPCRs have been implicated in a number of neuropsychiatric disorders. For example, CCK receptors, which are GPCRs found in peripheral tissues such as the pancreas, stomach, intestine and gall bladder, and, in limited amounts, in the brain, have been implicated in the pathogenesis of schizophrenia, Parkinson's disease, drug addiction and eating disorders. Aberrant expression of GPCRs have additionally been associated with the pathogenesis of inflammatory diseases, infectious diseases (e.g., such as AIDS), ocular blindness, cardiovascular diseases, and many other diseases and pathological conditions.

Because of the large numbers of diseases in which the aberrant expression of one or more GPCR pathway molecules has been implicated, GPCRs and other GPCR pathway molecules serve as promising drug targets. Genomic and proteomic information relating to GPCRs have been collected and organized in a web based system, the GPCRDB Information System which can be implemented by accessing the World Wide Web using the URL http://www.gpcr.org/7tm/. The GPCRDB system includes links to genomic databases, protein databases, drug databases, and various reference databases. The system includes sequence information, mutant data, and ligand binding constant information and provides computational alignment tools, three-dimensional models, phylogenetic trees and two dimensional visualization tools. The system does not link the various databases to clinical information.

### SUMMARY OF THE INVENTION

The physiological responses of an organism to a condition, (e.g., such as a disease, an environmental condition, exposure to a drug, and the like) involve the complex interactions of multiple genes. Thus, a single gene-single tissue analysis or even a multiple gene-single tissue analysis will rarely provide a true picture of how to treat perturbations in these responses. There is a need in the art for a system and method for identifying genes involved in molecular pathways and which can simulate the effects of changes in the expression of multiple interacting gene products to evaluate and predict physiological responses. In particular, there is a need in the art to characterize biomolecules involved in GPCR signaling pathways and to obtain molecular profiles of the expression of these biomolecules during physiological responses to diseases, drugs, environmental conditions and the like.

Accordingly, the invention provides tissue microarrays and a specimen-linked database for evaluating changes in the expression of GPCR pathway molecules in a patient in response a to one or more conditions. In one aspect, tissue microarrays are provided which comprise a plurality of tissue samples stably associated with different sublocations on a substrate. At least one biological characteristic of the tissue sample at each sublocation is known (e.g., such as tissue type, tissue source, and the like). The tissue microarray is identified by an identifier which links the tissue microarray to a tissue information system comprising a specimen-linked database and an information management system. The information management system comprises search and relationship determining functions enabling a user to search the database and to determine relationships between biological characteristics of tissues on the microarray (e.g., such as the expression of GPCR pathway biomolecules) and the biological characteristics of other tissues linked to the database (i.e., tissues included in other tissue microarrays for which data has been obtained and inputted into the database).

Preferably, the system enables a user to identify and validate relationships between the expression of GPCR pathway biomolecules in tissues samples in a plurality of micorarrays and the characteristics of patients who were the sources of these tissues. More preferably, the characteristics of patients being evaluated include the physiological responses of these patients to one or more conditions.

In one aspect, the tissue information system comprises at least one user device connectable to the network which displays an interface for entering an identifier identifying a tissue microarray. Entering the identifier into the interface enables the user to access the database and obtain information relating to tissue samples in the microarray. In a preferred aspect, entering the identifier causes a representation of the microarray identified by the identifier to be displayed on the interface. Selecting a representation of a sublocation on the array links the user to information relating to tissue at that sublocation on the microarray.

In one aspect, tissue microarrays are provided which comprise multiple tissue samples from one or more patients, i.e., tissue microarrays which are representative of the whole body of one or more patients. These “whole body microarrays” are used to evaluate the responses of multiple organ systems of one or more patients to a condition such as a disease, a drug, a toxic agent, an environmental condition, and combinations thereof. For example, the microarray can be reacted with at least one molecular probe which specifically binds to a GPCR pathway biomolecule and the reactivity of the at least one molecular probe can be used to determine the expression of the biomolecule in a plurality of different tissues. In this way, the effect of a condition on GPCR pathway biomolecules in an entire organism can be determined in a single assay. In preferred aspects, the response of the organism is monitored by evaluating the expression of multiple GPCR pathway biomolecules at a single time.

In one aspect, tissue microarrays according to the invention are used in conjunction with the tissue information system to identify and confirm relationships between biomolecules which are suspected of being are part of a GPCR pathway. For example, an absence of expression or a reduced or higher level of expression of a GPCR pathway molecule or the presence of a modified form of the GPCR pathway molecule in one or more tissues in one or more microarrays can be correlated by the tissue information system with a consistent lack of expression or reduced or higher level of expression or the presence of a particular modified form of one or more other biomolecules in the same samples on the same microarray (e.g., using differentially labeled probes), thereby identifying these other biomolecules as potentially belonging to the same GPCR pathway. Alternatively, or additionally, the expression of multiple biomolecules in different but identical microarrays (e.g., microarrays sectioned from a single recipient tissue block) can be evaluated using different probes labeled with the same type of label. By comparing data from multiple assays, the system can rank identified pathways according to the likelihood that they exist in vivo.

In one aspect, candidate GPCR pathway molecules are identified in both human and non-human animals and conserved GPCR pathway molecules are identified. In another aspect, non-human animals are provided which comprise disruptions in one or more genes responsible for the expression of one or more candidate pathway molecules and are used to generate microarray(s), such as a whole body tissue microarray. The expression of GPCR pathway molecules in tissues of such microarray(s) is used to verify predictions by the system that the expression of one or more biomolecules identified as belonging to the pathway will be altered by a disruption of the gene. The effect of restoring the function of the gene to an animal (e.g., by crossing the animal to a wild type background) can then be used to verify that the expression of other molecules in the GPCR pathway is similarly restored.

In one aspect, the impact of disease or a pathological condition on the physiological responses of an organism is evaluated. For example, a tissue microarray comprising samples from a patient having a disease or pathological condition can be reacted with one or more molecular probes, and preferably, with a plurality of molecular probes, which react specifically with one or more biomolecules in a GPCR pathway. The expression of at least one biomolecule reactive with the one or more molecular probes is then determined and the information is provided to the tissue information system and stored in the specimen-linked database. The system can then determine relationships between the expression of the at least one biomolecule and a patient's response to the disease or pathological condition. In preferred aspects, the system identifies biomolecules which are diagnostic or prognostic of the disease or pathological condition.

The invention also provides diagnostic assays in which the expression of one or more biomolecules in a tissue sample from a patient suspected of having a disease or pathological condition is determined and compared to the expression of biomolecules associated with disease using the specimen-linked database. For example, the tissue information system can be used to input data relating to the expression of GPCR pathway biomolecules in tissues from the patient suspected of having a disease or pathological condition, and the information management system can be used to provide an indication of the likelihood that the patient has the disease or the pathological condition. In one aspect, the system also provides information relating to treatment options.

In another aspect, the invention provides a specimen-linked database which comprises one or more subdatabases including information relating to tissue microarrays comprising samples from patients sharing one or more common characteristics. For example, the specimen-linked database can comprise an autopsy database with information relating to tissues obtained from autopsies, an oncology database comprising information relating to tissues obtained from cancer patients, a neurodegenerative disease database comprising information relating to tissues obtained from patients having a neurodegenerative disease, a neuropsychiatric disease database comprising information relating to tissues obtained from patients classified according to various DSM-IV criteria, a cardiovascular disease database, a gastrointestinal disease database, and the like. In a preferred aspect, the tissue information system uses information in these various databases to simulate GPCR pathways comprising biomolecules having a strong likelihood of being affected in patients with a disease such as cancer or a neurodegenerative or neuropsychiatric disease, and the like.

In still another aspect, the impact of a drug on the physiological responses of an organism can be evaluated. For example, a whole body tissue microarray comprising samples from a patient treated with a drug can be reacted with one or more molecular probes, and preferably with a plurality of molecular probes, which react specifically with one or more biomolecules in a GPCR pathway. The expression of the one or more biomolecules in the pathway can be determined and information relating to this expression can be provided to the tissue information system. The system can then identify relationships between the expression of the one or more biomolecules in treated patients with the expression of the one or more biomolecules in untreated patients, or in patients treated with different doses, or for different amounts of time, with the drug. The system can preferably be used to predict the impact of changes in the expression of the one or more biomolecules on the expression of other biomolecules in the pathway. Still more preferably, the system is used to identify drugs with minimal adverse affects by identifying drugs which have the least effect on molecular pathways in non-diseased tissues on the microarray. Because of the large numbers of microarrays which can be evaluated in parallel, the effect of concurrent exposure to a plurality of drugs can be evaluated and/or the effects of underlying conditions or concurrent illnesses.

In another aspect, tissue microarrays are used to evaluate the toxicity of an agent or to evaluate the impact of one or more environmental conditions on the physiological responses of an organism. As above, information relating to the expression of GPCR pathway biomolecules in one or more tissues from an organism which has been exposed to the agent or condition can be obtained by probing one or more tissue microarrays from such an organism and adding the information to a specimen-linked database. Using an information management system coupled to the database, the user can identify and validate possible relationships between an expression pattern observed and pathological effects. Accordingly, in one aspect, the tissue information system is used to rank agents or conditions according to their likely toxic effects. In some aspects, tissues arrayed on the microarrays are obtained from different developmental stages of a developing organism whose parent has been exposed to an agent or condition, and the teratogenic effects of the agent or condition are determined.

### BRIEF DESCRIPTION OF THE DRAWINGS

The objects and features of the invention can be better understood with reference to the following detailed description and accompanying drawings.

FIG. 1 shows a flow chart according to one aspect of the invention in which tissue microarrays according to the invention are used in conjunction with gene chips to identify, prioritize, and validate drug targets.

FIG. 2A is a schematic of a microarray according to one aspect of the invention. FIG. 2B is a schematic of a profile array substrate according to one aspect of the invention comprising a microarray. FIG. 2C shows a mixed format microarray comprising a large format array and small format array on a single substrate.

FIG. 3 is a schematic diagram illustrating a system comprising a specimen-linked database and information management system according to one aspect of the invention.

FIG. 4 is a flow chart showing a method according to one aspect of the invention, for organizing and displaying tissue information obtained from a tissue microarray.

FIGS. 5A-E show interfaces on the display of a user device connectable to the network for organizing a displaying information relating to tissue microarrays.

FIG. 6 shows an optical system according to one aspect of the invention for detecting and processing optical information from a tissue microarray.

FIG. 7 illustrates an interface on a display of a user device, according to one aspect, for accessing a genomics medicine database in the system.

FIG. 8 illustrates an interface on a display of a user device, according to one aspect, displaying relationships identified by the system.

FIG. 9 is a flow chart showing a method of validating information included in the database.

FIGS. 10A-C shows a display of a user device according to one aspect displaying information in the database from a plurality of molecular profiling experiments.

### DETAILED DESCRIPTION

The invention relates to a method and system for identifying and evaluating the physiological responses of an organism to a condition, such as a disease or other pathological condition, a drug or agent, an environmental condition, and the like, by evaluating the expression of one or more GPCR pathway biomolecules in tissue microarrays from a plurality of patients. In one aspect, a tissue information system is provided comprising a specimen-linked database and an information management system for accessing, organizing, and displaying tissue information obtained from tissue microarrays. Preferably, the system is used to model and validate GPCR pathways affected during one or more physiological responses to a condition.

The following definitions are provided for specific terms which are used in the following written description.

As used herein, the term “information about the patient” refers to any information known about the individual (a human or non-human animal) from whom a tissue sample was obtained. The term “patient” does not necessarily imply that the individual has ever been hospitalized or received medical treatment prior to obtaining a tissue sample. The term “patient information” includes, but is not limited to, age, sex, weight, height, ethnic background, occupation, environment, family medical background, the patient's own medical history (e.g., information pertaining to prior diseases, diagnostic and prognostic test results, drug exposure or exposure to other therapeutic agents, responses to drug exposure or exposure to other therapeutic agents, results of treatment regimens, their success, or failure, history of alcoholism, drug or tobacco use, cause of death, and the like). The term “patient information” refers to information about a single individual. Information from multiple patients provides “demographic information,” defined as statistical information relating to populations of patients, organized by geographic area or other selection criteria, while “epidemiological information” is defined as information relating to the incidence of disease in populations.

As defined herein, the term “information relating to” is information which summarizes, reports, provides an account of, and/or communicates particular facts, and in some aspects, includes information as to how facts were obtained and/or analyzed.

As used herein, the term, “in communication with” refers to the ability of a system or component of a system to receive input data from another system or component of a system and to provide an output in response to the input data. “Output” may be in the form of data or may be in the form of an action taken by the system or component of the system.

As used herein, the term “provide” means to furnish, supply, or to make available.

As defined herein, a “tissue” is an aggregate of cells that perform a particular function in an organism. The term “tissue” as used herein refers to cellular material from a particular physiological region. The cells in a particular tissue may comprise several different cell types. A non-limiting example of this would be brain tissue that further comprises neurons and glial cells, as well as capillary endothelial cells and blood cells. The term “tissue” also is intended to encompass a plurality of cells contained in a sublocation on the tissue microarray that may normally exist as independent or non-adherent cells in the organism, for example immune cells, or blood cells. The term is further intended to encompass cell lines and other sources of cellular material that now exist which represent specific tissue types (e.g., by virtue of expression of biomolecules characteristic of specific tissue types).

As defined herein, a “molecular probe” is any detectable molecule, or is a molecule which produces a detectable molecule upon reacting with a biological molecule. “Reacting” encompasses binding, labeling, or catalyzing an enzymatic reaction. A “biological molecule” or “biomolecule” is any molecule which is found in a cell or within the body of an organism.

As used herein, the term “biological characteristics of a tissue” refers to the phenotype and genotype of the tissue or cells within a tissue, and includes tissue type, morphological features; the expression of biological molecules within the tissue (e.g., such as the expression and accumulation of RNA sequences, the expression and accumulation of proteins (including the expression of their modified, cleaved, or processed forms, and further including the expression and accumulation of enzymes, their substrates, products, and intermediates); and the expression and accumulation of metabolites, carbohydrates, lipids, and the like). A biological characteristic can also be the ability of a tissue to bind, incorporate, or respond to a drug or agent. “Biological characteristics of a tissue source” are the characteristics of the organism which is the source of the tissue (e.g., such as the age, sex, and physiological state of the organism) and encompasses patient information.

As defined herein, “a diagnostic trait” is an identifying characteristic, or set of characteristics, which in totality, are diagnostic. The term “trait” encompasses both biological characteristics and experiences (e.g., exposure to a drug, occupation, place of residence). In one aspect, a trait is a marker for a particular cell type, such as a transformed, immortalized, pre-cancerous, or cancerous cell, or a state (e.g., a disease) and detection of the trait provides a reliable indicia that the sample comprises that cell type or state. Screening for an agent affecting a trait thus refers to identifying an agent which can cause a detectable change or response in that trait which is statistically significant within 95% confidence levels.

As used herein, the term “expression” refers to a level, form, or localization of a product. For example, “expression of a protein” refers to any or all of the level, form (e.g., presence, absence, or quantity of modifications, or cleavage or other processed products), or localization (e.g., subcellular and/or extracellular compartment) of the protein.

A “disease or pathology” is a change in one or more biological characteristics that impairs normal functioning of a cell, tissue, and/or organism. A “pathological condition” encompasses a disease but also encompasses abnormal responses which are not associated with any particular infectious organism or single genetic alteration in an individual. For example, as defined herein, a stroke or an immune response occurring after transplantation of an organism would be encompassed by the term “pathological condition.”

As used herein, the term “cancer” refers to a malignant disease caused or characterized by the proliferation of cells which have lost susceptibility to normal growth control. “Malignant disease” refers to a disease caused by cells that have gained the ability to invade either the tissue of origin or to travel to sites removed from the tissue of origin.

As used herein, the term “difference in biological characteristics” refers to an increase or decrease in a measurable expression of a given biological characteristic. A difference may be an increase or a decrease in a quantitative measure (e.g., amount of a protein or RNA encoding the protein) or a change in a qualitative measure (e.g., location of the protein). Where a difference is observed in a quantitative measure, the difference according to the invention will be at least about 10% greater or less than the level in a normal standard sample. Where a difference is an increase, the increase may be as much as about 20%, 30%, 50%, 70%, 90%, 100% (2-fold) or more, up to and including about 5-fold, 10-fold, 20-fold, 50-fold or more. Where a difference is a decrease, the decrease may be as much as about 20%, 30%, 50%, 70%, 90%, 95%, 98%, 99% or even up to and including 100% (no specific protein or RNA present). It should be noted that even qualitative differences may be represented in quantitative terms if desired. For example, a change in the intracellular localization of a polypeptide may be represented as a change in the percentage of cells showing the original localization.

As defined herein, the “efficacy of a drug” or the “efficacy of a therapeutic agent” is defined as ability of the drug or therapeutic agent to restore the expression of diagnostic trait to values not significantly different from normal (as determined by routine statistical methods, to within 95% confidence levels).

As defined herein, “a tissue microarray” is a microarray that comprises a plurality of sublocations, each sublocation comprising tissue cells and/or extracellular materials from tissues, or cells typically infiltrating tissues, where the morphological features of the cells or extracellular materials at each sublocation are visible through microscopic examination. The term “microarray” implies no upper limit on the size of the tissue sample on the array, but merely encompasses a plurality of tissue samples which, in one aspect, can be viewed using a microscope.

As defined herein, “a whole body tissue microarray” is a microarray comprising tissue samples representing the whole body of an organism. In one aspect, the microarray comprises at least about five different tissue samples from an organism, at least about ten different tissues from an organism, or at least about 20 different tissues from an organism. For example, in one aspect, a whole body microarray comprises at least about five different tissues selected from the group consisting of brain tissue, cardiac tissue, liver tissue, pancreatic tissue, spleen tissue, stomach tissue, lung tissue, skin tissue, eye tissue, colon tissue, reproductive organ tissue, and kidney tissue. In preferred aspects, a sample of a bodily fluid is also included, such as a blood sample, lymph sample, CSF sample, and the like.

As defined herein a “a sample” is a material suspected of comprising an analyte and includes a biological fluid, suspension, buffer, collection of cells, scraping, fragment or slice of tissue. A biological fluid includes blood, plasma, sputum, urine, cerebrospinal fluid, lavages, and leukophoresis samples.

As used herein “donor block” refers to an embedding material comprising a tissue or cell(s). While referred to as a “block”, the embedded tissue or cell(s) can be generally of any shape or size so long as an at least about 0.3 mm in diameter sample core can be obtained from it. A sample from a donor block can be placed directly onto a slide or can be placed in a recipient block.

As used herein “donor sample” refers to an embedded tissue or cell sample obtained from the donor block.

As used herein “recipient block” refers to a block formed from a fast-freezing embedding material which is capable of holding frozen donor samples in a pattern so that the location of the frozen donor samples relative to each other is maintained when the frozen block is sectioned to produce an array of frozen tissue and/or cell samples. The term “microarray block” refers more specifically to a recipient block which comprises a desired number of frozen donor samples.

As used herein a “tissue” is an aggregate of cells that perform a particular function in an organism and generally refers to cells and cellular material (e.g., such as extracellular matrix material) from a particular physiological region. The cells in a particular tissue can comprise several different cell types. A non-limiting example of this would be brain tissue that further comprises neurons and glial cells, as well as capillary endothelial cells and blood cells.

As used herein a “nucleic acid microarray,” a “peptide microarray,” a “polypeptide microarray,” a “protein microarray,” or a “small molecule microarray” or “arrays” of any of nucleic acids, peptides, polypeptides, proteins, small molecules, refer to a plurality of nucleic acids, peptides, polypeptides, proteins, or small molecules, respectively, that are immobilized on a substrate in assigned locations (i.e., known locations).

As used herein “a tissue microarray” is a microarray that comprises a plurality of sublocations, each sublocation comprising tissue cells and/or extracellular materials from tissues, or cells typically infiltrating tissues, where the morphological features of the cells or extracellular materials at each sublocation are visible through microscopic examination. The term “microarray” implies no upper limit on the size of the tissue sample on the microarray, but merely encompasses a plurality of tissue samples which, in one aspect, can be viewed using a microscope.

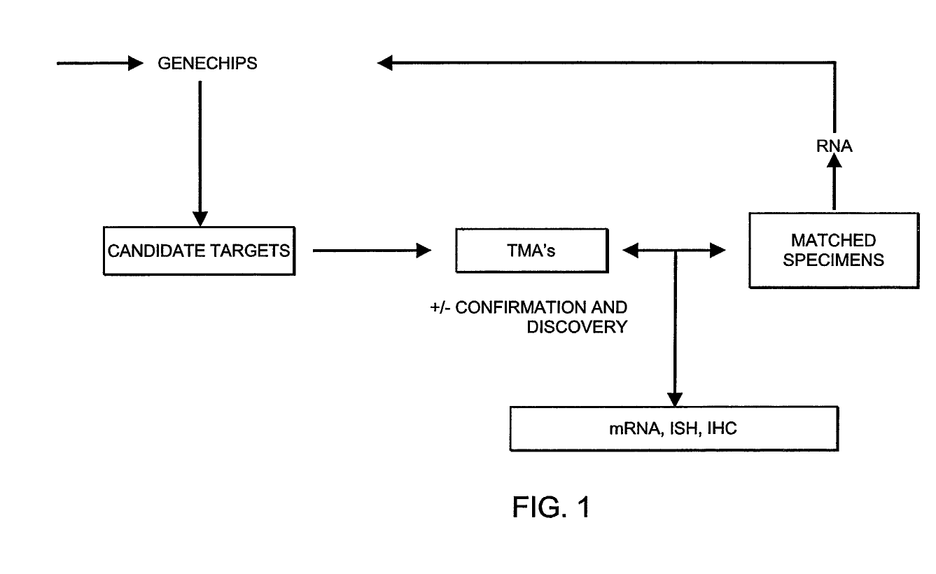

As used herein a “large format microarray” comprises at least one sublocation comprising at least two different cell types (e.g., abnormally growing cells and normally growing cells, such as cancer cells and non-cancer cells), at least one cell type and extracellular matrix material, or a plurality of cells comprising at least one cell expressing a heterogeneously expressed biological characteristic (e.g., a biological characteristic expressed in less than 80% of cells of a given tissue or cell type). In one aspect, a large format tissue microarray comprises at least one sublocation being larger than 0.6 mm in at least one dimension. In contrast, a “small format” microarray comprises samples of about 0.6 mm in diameter and an “ultrasmall format” microarray comprises tissue samples less than about 0.6 mm in diameter (e.g., preferably, about 0.3 mm in diameter). “Mixed format” arrays comprise samples of varying sizes and include two or more of small format samples, large format samples, and ultrasmall format samples (see, e.g., FIG. 1C).

As used herein a “microarray sample” or “sample” refers to either a tissue sample or cell sample, unless specifically used in connection with the terms “nucleic acid microarray”, “polypeptide array”, “peptide array” or “small molecule” array. A sample is a material suspected of containing one or more cellular or extracellular structures and includes a biological fluid, suspension, buffer, collection of cells, a scraping, fragment, smear, or slice of tissue. A biological fluid includes, but is not limited to blood, plasma, sputum, urine, amniotic fluid, lavages and leukophoresis samples.

As used herein “a portion of a donor sample” is a section through a donor sample.

As used herein, a portion of a sample which is “stably” associated with a substrate refers to a portion which does not substantially move from its position on the substrate during one or more molecular procedures.

As used herein “a cell sample” is distinguished from a tissue sample in that it comprises a cell or cell which is disassociated from other cells.

As used herein “a hole sized to receive a donor” sample refers to a hole in the recipient block which fits a donor sample snugly, so that there is no appreciable space between the donor sample and the walls of the hole (e.g., less than about 1 mm between the edge of a donor sample and the walls of the hole in the recipient block).

As used herein “different types of tissues” refers to tissues which are preferably from different organs or which are at least from anatomically and histologically distinct sites in the same organ.

As used herein “information relating to the location of each donor sample” is information which includes at least the coordinates of the donor sample in the block.

As used herein “substantially identical microarrays” refer to microarrays obtained by sectioning a single microarray block. Preferably, substantially identical microarrays comprise sections which are within about 0-500 μm of each other in a microarray block. Substantially identical microarrays comprise a one-to-one correspondence of samples, such that samples at identical coordinates in each of a plurality of microarrays will be substantially identical.

As used herein “coordinates” refer to the x, y location of a sample in a microarray comprising samples arranged in rows and columns, wherein the x coordinate refers to the column number of the sample and the y coordinate refers to the row number of the sample.

As used herein “substantially intact morphological features” refers to features which at least can be viewed under a microscope to distinguish subcellular features (e.g., such as a nucleus, an intact cell membrane, organelles, and/or other cytological features).

As used herein “molecular procedure” refers to contact with a test reagent or molecular probe such as an antibody, nucleic acid probe, enzyme, chromagen, label, and the like. In one aspect, a molecular procedure comprises one or more of a plurality of hybridizations, incubations, fixation steps, changes of temperature (from about −4° C. to about 100° C.), exposures to solvents, and/or wash steps.

As used herein “similar demographic characteristics” or “demographically matched”, refers to patients who minimally share the same sex and belong to the same age grouping (e.g., are within about 5 to fifteen years of a selected age). Additional shared characteristics can be selected, including, but not limited to, shared place of residence (e.g., within a hundred mile radius of a particular location), shared occupation, shared history of illnesses, shared ethnic background, and the like.

As defined herein, a “database” is a collection of information or facts organized according to a data model which determines whether the data is ordered using linked files, hierarchically, according to relational tables, or according to some other model determined by the system operator. The organization scheme that the database uses is not critical to performing the invention, so long as information within the database is accessible to the user through an information management system. Data in the database are stored in a format consistent with an interpretation based on definitions established by the system operator (i.e., the system operator determines the fields which are used to define patient information, molecular profiling information, or another type of information category). As used herein, a “specimen-linked database” is a database which cross-references information in the database to tissue specimens provided on one or more microarrays, and preferably using codes, such as SNOMED® codes, ICD-9 codes, and/or DSM-IV TR codes.

As defined herein, “a system operator” is an individual who controls access to the database.

As used herein, the term “information management system” refers to a system which comprises a plurality of functions for accessing and managing information within the database. Minimally, an information management system according to the invention comprises a search function, for locating information within the database and for displaying a least a portion of this information to a user, and a relationship determining function, for identifying relationships between information or facts stored in the database.

As defined herein, an “interface” or “user interface” or “graphical user interface” is a display (comprising text and/or graphical information) displayed by the screen or monitor of a user device connectable to the network which enables a user to interact with the database and information management system according to the invention.

As used herein, the term “link” refers to a point-and-click mechanism implemented on a user device connectable to the network which allows a viewer to link (or jump) from one display or interface where information is referred to (“a link source”), to other screen displays where more information exists (a “link destination”). The term “link” encompasses both the display element that indicates that the information is available and a program which finds the information (e.g., within the database) and displays it one the destination screen. In one aspect, a link is associated with text; however, in other aspects, links are associated with images or icons. In some aspects, selecting a link (e.g., by right clicking using a mouse) will cause a drop down menu to be displayed which provides a user with the option of viewing one of several interfaces. Links can also be provided in the form of action buttons, radiobuttons, check buttons and the like.

As defined herein, a “browser” is a program which supports the displaying of documents, across a network. Browsers enable accessing linked information over the Internet and other networks, as well as from magnetic disk, CD-ROM, or other memory sources.

The term “providing access to at least a portion of a database” as defined herein refers to making information in the database available to user(s) through a visual or auditory means of communication.

As used herein, “through a visual means of communication” includes displaying or providing written text, image(s), or a combination of written and graphical information to a user of the database.

As used herein, “through an auditory or verbal means of communication” refers to providing the user with taped audio information, or access to another user who can communication the information through speech or sign language. Written and/or graphical information can be communicated through a printed report or electronically (e.g., through a display on the display of a computer or other processor, through email or other electronic messaging systems, through a wireless communications device, via facsimile, and the like). Access can be unrestricted or restricted to specific subdatabases within the database.

As used herein, “instruction pipelining” refers to the sequence of bus operations that occurs during instruction execution. The instruction-fetch, decode, operand-fetch, execute pipeline is essentially invisible to the user, except in some cases where the pipeline must be broken (such as for branch instructions). In the operation of the pipeline the instruction fetch, decode, operand fetch, and execute operations are independent which allow instruction executions to overlap. Thus, during any given cycle of operations, one to n different instructions can be active, each at a different stage of completion, resulting in one to n-deep pipeline (see, e.g., as described in U.S. Pat. No. 5,724,248, the entirety of which is incorporated by reference herein.

As used herein, “pathway molecules” or “pathway biomolecules” are molecules involved in the same pathway and whose accumulation and/or activity and/or form (i.e., referred to collectively as the “expression” of a molecule) is dependent on other pathway molecules, or whose accumulation and/or activity and/or form affects the accumulation and/or activity or form of other pathway target molecules. For example, a “GPCR pathway molecule” is a molecule whose expression is affected by the interaction of a GPCR and its cognate ligand (a ligand which specifically binds to a GPCR and which triggers a signaling response, such as a rise in intracellular calcium). Thus, a GPCR itself is a GPCR pathway molecule, as is its ligand, as is intracellular calcium. An “early pathway molecule” is a molecule whose expression is required for the expression of at least about five other genes, while a “late pathway” molecule is a molecule whose expression is required for the expression of about two or fewer other genes.

As used herein “a correlation” refers to a statistically significant relationship determined using routine statistical methods known in the art. For example, in one aspect, statistical significance is determined using a Student's unpaired t-test, considering differences as statistically significant at p<0.05.

As used herein a “diagnostic probe” is a probe whose binding to a tissue and/or cell sample provides an indication of the presence or absence of a particular trait. In one aspect, a probe is considered diagnostic if it binds to a diseased tissue and/or cell (“disease samples”) in at least about 80% of samples tested comprising diseased tissue/cells and binds to less than 10% of non-diseased tissue/cells in samples (“non-disease” samples). Preferably, the probe binds to at least about 90% or at least about 95% of disease samples and binds to less than about 5% or 1% of non-disease samples.

As used herein “electronic subtraction” refers to a method of comparing a first expressed sequence database with a second expressed sequence database and electronically removing sequences which are in both the first and second database. Methods of electronic subtraction are described in U.S. Pat. No. 5,840,484, for example, the entirety of which is incorporated by reference herein.

As used herein “a probe corresponding to a differentially expressed sequences” is a probe capable of specifically reacting with the sequence such that reactivity of the probe with a sample indicates the presence of the sequence.

Tissue Microarrays

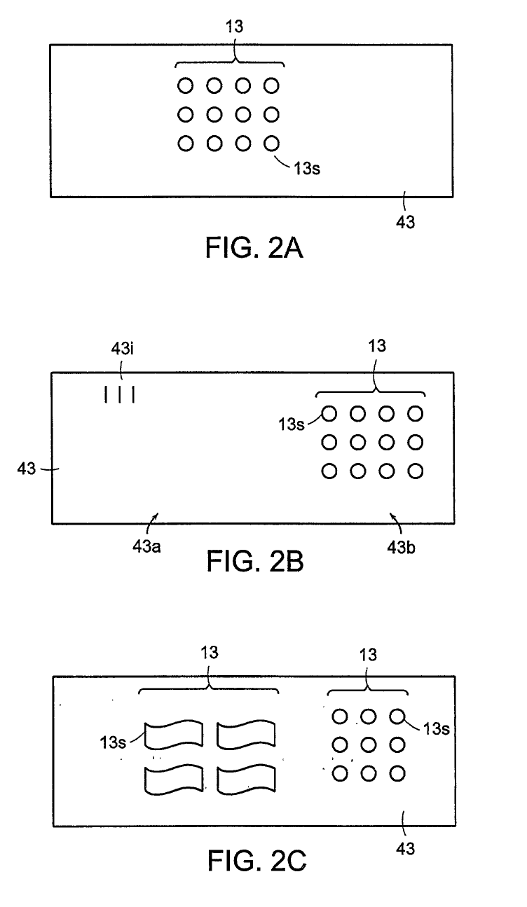

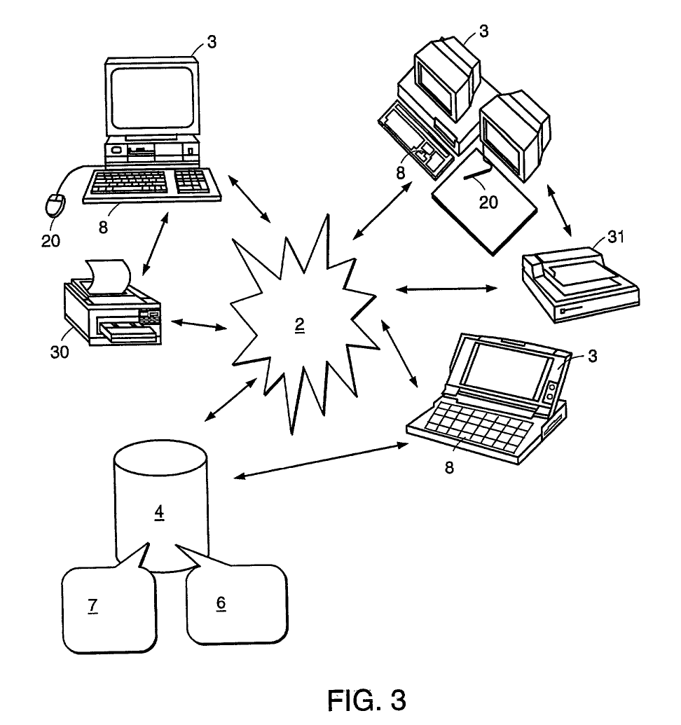

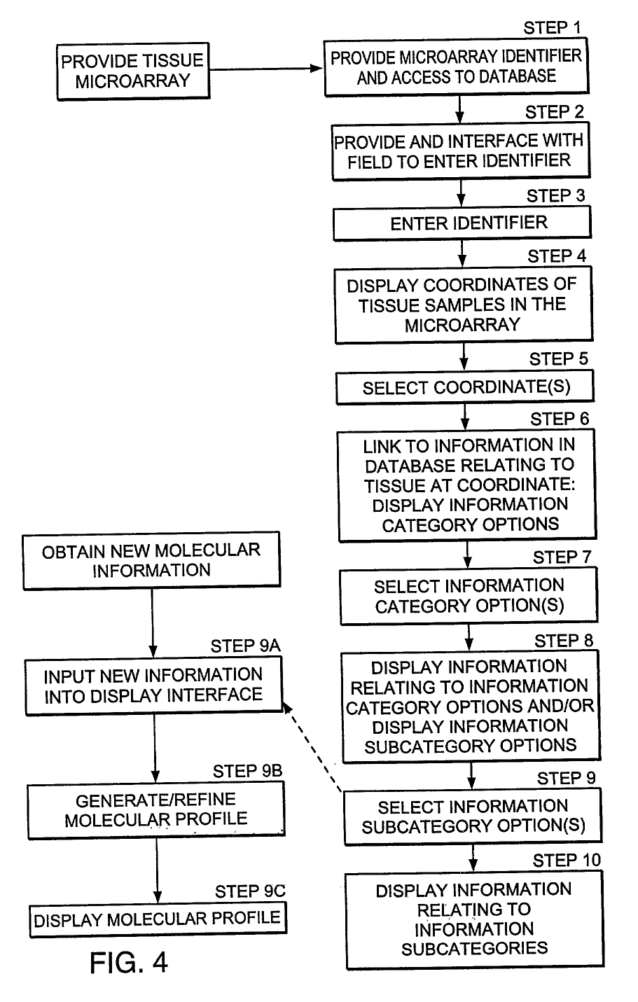

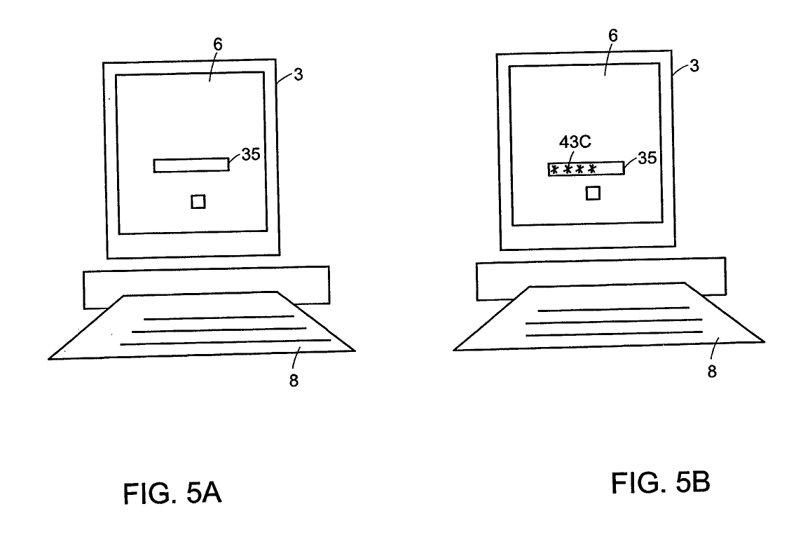

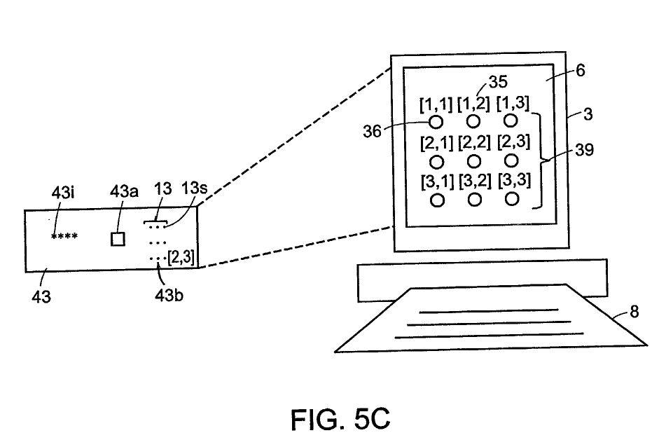

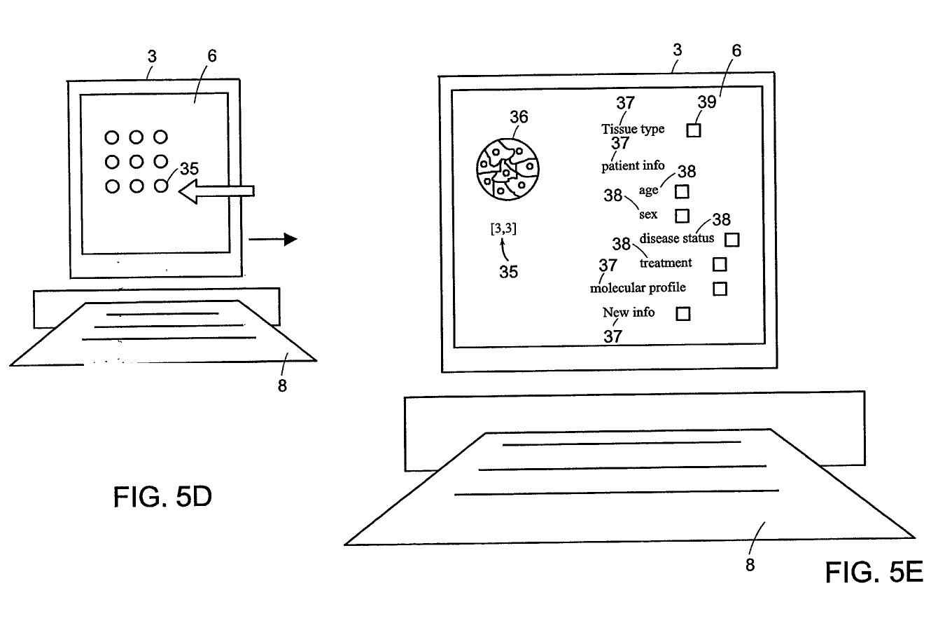

As shown in FIG. 2A, microarrays 13 according to the invention comprise a plurality of sublocations 13s, each sublocation comprising a tissue sample having at least one known biological characteristic (e.g., such as tissue type). In one aspect, the tissue sample at at least one sublocation 13s has substantially intact morphological features which can be at least viewed under a microscope to distinguish subcellular features (e.g., such as a nucleus, an intact cell membrane, organelles, and/or other cytological features), i.e., the tissue is not lysed (see FIG. 2C and FIG. 3, for example).

In one aspect of the invention, the microarray comprises a substrate 43 to facilitate handling of the microarray 13 through a variety of molecular procedures. As used herein, “molecular procedure” refers to contact with a test reagent or molecular probe such as an antibody, nucleic acid probe, enzyme, chromagen, label, and the like. In one aspect, a molecular procedure comprises a one or more of a plurality of hybridizations, incubations, fixation steps, changes of temperature (from −4° C. to 100° C.), exposures to solvents, and/or wash steps. Suitable substrates are described in U.S. patent application Ser. No. 09/781,016, filed Feb. 9, 2001, the entirety of which is incorporated herein by reference.

In one aspect of the invention, shown in FIG. 2B the substrate 43 is a “profile array substrate” designed to accommodate a control tissue microarray and a test tissue or cell sample for comparison with the control tissue microarray. In this aspect, the substrate 43 comprises a first location 43a and a second location 43b. The first location 43a is for placing a test tissue sample, while the second sublocation 43b comprises the microarray 13. This profile microarray substrate 43 allows testing of a test tissue sample to be done simultaneously with the testing of tissue samples on the microarray 13 having at least one known biological characteristic allowing for a side-by-side comparison of biological characteristics expressed in the test sample with the characteristics of the tissues in the microarray 13. Profile microarray substrates 43 are disclosed in U.S. Provisional Application Serial No. 60/234,493, filed Sep. 22, 2000, the entirety of which is incorporated by reference herein.

In one aspect of the invention, as shown in FIG. 2B, the substrate 43 comprises a location for placing an identifier 43i (e.g., a wax pencil or crayon mark, an etched mark, a label, a bar code, a microchip for transmitting radio or electronic signals, and the like). For example, the identifier can be a microchip which communicates with a processor which comprises, or can access, stored information relating to the identity and address of sublocations 13s on the microarray and/or including patient information regarding the individual from whom the tissue was taken.

Sources of Samples

In one aspect, the microarray samples are tissue samples. Tissue samples can be obtained from cadavers or from patients who have recently died (e.g., from autopsies). Tissues also can be obtained from surgical specimens, pathology specimens (e.g., biopsies), from samples which represent “clinical waste” which would ordinarily be discarded from other procedures. Samples can be obtained from adults, children, and/or fetuses (e.g., from elective abortions or miscarriages).

Cells also can be obtained to provide one or more samples in the microarray. Cells can be obtained from suspensions of cells from tissues (e.g., from a suspension of minced tissue cells, such as from a dissected tissue), from bodily fluids (e.g., blood, plasma, sera, and the like), from mucosal scrapings (e.g., such as from buccal scrapings or pap smears), and/or from other procedures such as bronchial ravages, amniocentesis procedures and/or leukophoresis. In some aspects, cells are cultured first prior to being made part of the microarray to expand a population of cells to be analyzed. Cells from continuously growing cell lines, from primary cell lines, and/or stem cells, also can be used.

In one aspect, a microarray 13 comprises a plurality of tissues/cells from a single individual, i.e., the microarray represents the “whole body” of an individual. Preferably, a “whole body microarray” according to the invention comprises at least five different types of tissues from a single patient. More preferably, the whole body microarray comprises at least 10 or at least 15 different tissues. Tissues can be selected from the group consisting of: skin, neural tissue, cardiac tissue, liver tissue, stomach tissue, large intestine tissue, colon tissue, small intestine tissue, esophagus tissue, lung tissue, cardiac tissue, spleen tissue, pancreas tissue, kidney tissue, tissue from a reproductive organ(s) (male or female), adrenal tissue, and the like. Tissues from different anatomic or histological locations of a single organ can also be obtained, e.g., such as from the cerebellum, cerebrum, and medulla, where the organ is the brain. Some microarrays comprise samples representative of organ systems (i.e., comprising samples from multiple organs within an organ system), e.g., the respiratory system, urinary system, kidney system, cardiovascular system, digestive system, and reproductive system (male or female). In a preferred aspect, a whole body microarray additionally comprises a sample of cells from a bodily fluid of the patient (e.g., from a blood sample).

The microarray 13 also can comprise a plurality of sublocations 13s comprising cells from individuals sharing a trait. For example, the trait shared can be gender, age, pathology, predisposition to a pathology, exposure to an infectious disease (e.g., HIV), kinship, death from the same disease, treatment with the same drug, exposure to chemotherapy, exposure to radiotherapy, exposure to hormone therapy, exposure to surgery, exposure to the same environmental condition (e.g., such as carcinogens, pollutants, asbestos, TCE, perchlorate, benzene, chloroform, nicotine and the like), the same genetic alteration or group of alterations, expression of the same gene or sets of genes (e.g., samples can be from individuals sharing a common haplotype, such as a particular set of HLA alleles), and the like. In another aspect of the invention, the microarray 13 is a reflection of a plurality of traits representing a particular patient demographic group of interest, e.g., overweight smokers, diabetics with peripheral vascular disease, individuals having a particular predisposition to disease (e.g., to sickle cell anemia, Tay Sachs, severe combined immunodeficiency, and the like).

Samples can be obtained from an individual with a disease or pathological condition, including, but not limited to: a blood disorder, blood lipid disease, autoimmune disease, bone or joint disorder, a cardiovascular disorder, respiratory disease, endocrine disorder, immune disorder, infectious disease, muscle wasting and whole body wasting disorder, neurological disorders including neurodegenerative and/or neuropsychiatric diseases, skin disorder, kidney disease, scleroderma, stroke, hereditary hemorrhage telangiectasia, diabetes, disorders associated with diabetes (e.g., PVD), hypertension, Gaucher's disease, cystic fibrosis, sickle cell anemia, liver disease, pancreatic disease, eye, ear, nose and/or throat disease, diseases affecting the reproductive organs, gastrointestinal diseases (including diseases of the colon, diseases of the spleen, appendix, gall bladder, and others) and the like. For further discussion of human diseases, see Mendelian Inheritance in Man: A Catalog of Human Genes and Genetic Disorders by Victor A. McKusick (12th Edition (3 volume set) June 1998, Johns Hopkins University Press, ISBN: 0801857422), the entirety of which is incorporated herein. Preferably, samples from a normal demographically matched individual and/or from a non-disease tissue from a patient having the disease are arrayed on the same or a different microarray to provide controls.

In another aspect, microarrays are provided which comprise tissue samples from patients suffering from a neurodegenerative disease, i.e., a disease which causes progressive cell damage of neurons within the central nervous system (CNS) leading to loss of neuronal activity and cell death. Neurodegenerative diseases encompassed within the scope of the invention encompass chronic neurodegenerative diseases, including, but not limited to: AIDS dementia complex, demyelinating diseases, such as multiple sclerosis and acute transverse myelitis; extrapyramidal and cerebellar disorders' such as lesions of the corticospinal system; disorders of the basal ganglia or cerebellar disorders; hyperkinetic movement disorders such as Huntington's Chorea and senile chorea; drug-induced movement disorders, such as those induced by drugs which block CNS dopamine receptors; hypokinetic movement disorders, such as Parkinson's disease; Progressive supra-nucleo Palsy; structural lesions of the cerebellum; spinocerebellar degenerations, such as spinal ataxia, Friedreich's ataxia, cerebellar cortical degenerations, multiple systems degenerations (Mencel, Dejerine-Thomas, Shi-Drager, and Machado-Joseph); systemic disorders (Refsum's disease, abetalipoprotemia, ataxia, telangiectasia, and mitochondrial multi-system disorder); demyelinating core disorders, such as multiple sclerosis, acute transverse myelitis; and disorders of the motor unit such as neurogenic muscular atrophies (anterior horn cell degeneration, such as amyotrophic lateral sclerosis, primary lateral sclerosis, infantile spinal muscular atrophy and juvenile spinal muscular atrophy); Alzheimer's disease; Down's Syndrome in middle age; Diffuse Lewy body disease; Senile Dementia of Lewy body type; Weruicke-Korsakoff syndrome; chronic alcoholism; Creutzfeldt-Jakob disease; Subacute sclerosing panencephalitis Hallerrorden-Spatz disease; and Dementia pugilistica, diabetic peripheral neuropathy. (see, e.g., Berkow et al, eds., The Merck Manual, 16th edition, Merck and Co., Rahway, N.J., 1992, which reference, and references cited therein, are entirely incorporated herein by reference). Acute neurodegenerative diseases are also encompassed within the scope of the invention, such as conditions arising from stroke, schizophrenia, cerebral ischemia resulting from surgery and epilepsy as well as hypoglycemia and trauma resulting in injury of the brain, peripheral nerves or spinal cord, and the like.

In a further aspect, microarrays are provided which comprise tissue samples from patients who have a neuropsychiatric disorder. Such disorders include, but are not limited to, mental retardation, a learning disorder, a motor skills disorder, a communication disorder, a pervasive developmental disorder (e.g., autism, childhood disintegrative disorder, Rett's disorder), attention deficit and disruptive behavior disorders, eating disorders, tic disorders, elimination disorders (encopresis, enurisis), selective mutism, separation anxiety disorder, reactive attachment disorder of infancy or early childhood, delirium, dementia, amnestic disorders, cognitive disorders, catatonic disorder, personality change disorder, substance dependence or other substance induced disorders (e.g., a drug or alcohol abuse related disorder), schizophrenia (e.g., catatonic, disorganized, paranoid, residual, undifferentiated), schizophreniform disorder, delusional disorder, brief psychotic disorder, shared psychotic disorder, psychotic disorder due to a general medical condition (e.g., delusions, hallucinations), a substance-induced psychotic disorder, mood episodes (major depressive episode, hypomanic episode, manic episode, mixed episode), depressive disorders, bipolar disorders, acute stress disorder, agoraphobia, anxiety disorder, obsessive-compulsive disorder, panic disorder with or without agoraphobia, postraumatic stress disorder, obsessive-compulsive disorder, body dysmorphic disorder, conversion disorder, hypochondriasis, and other somatoform disorders, a dissociative disorder, a sexual or gender identity disorder, an eating disorder (e.g., anorexia, bulimia nervosa), a sleep disorder, kleptomania, pyromania, pathological gambling, intermittent explosive disorder, an Axis II personality disorder (each disorder as classified using DSM-IV criteria).

In one aspect, sets of microarrays 13 are provided representing multiple individuals with approximately 30,000 specimens covering at least about 1, 2, 5, 10, 15, 20, 25, 30, 40, or 50, different disease categories, including, but not limited to, any of the disease categories identified above. In some aspects, microarrays comprise samples from individuals have more than one disease condition (e.g., stroke and cardiovascular disease) and from individuals with only one of each of the diseases (e.g., samples from stroke patients without cardiovascular disease and samples from patients with cardiovascular disease but who have not experienced stroke). In some aspects, samples are from individuals with a chronic disease (e.g., such as Crohn's disease) and samples on the array include samples from patients in a remission period as well as samples from patients in an exacerbation period.

In one aspect, the microarray 13 comprises at least one sublocation 13s comprising cells from a single patient which are the target of a disease or pathology and comprises a plurality of sublocations 13s comprising cells from other tissues and organs from the same patient. In a further aspect of the invention, each sublocation 13s of the microarray comprises cells from different members of a pedigree sharing a family history of disease or susceptibility to a pathological condition (e.g., such as stroke), selected from the group consisting of siblings, twins, cousins, mothers, fathers, grandmothers, grandfathers, uncles, aunts, and the like. In another aspect of the invention, the “pedigree microarray” comprises environment-matched controls (e.g., husbands, wives, adopted children, step-parents, and the like).

In a preferred aspect, a microarray 13 is provided comprising a plurality of sublocations 13s which represent different stages of a cell proliferative disorder, such as cancer. In one aspect, in addition to including samples which comprise the primary target of the disease (e.g., such as tumor samples), the microarray 13 includes samples representing metastases of a cancer to secondary tissues/cells. Preferably, the microarray 13 also comprises normal tissues from the same patient from whom the abnormally proliferating tissue was obtained. A microarray can also be provided which comprises cells or tissues representing different stages of the cell cycle and may optionally include one ore more samples of cells from a patient with a cell proliferative disease or from a cell line which comprises abnormally proliferating cells (e.g., such as cancer cells). Cell lines can be developed from isolated cancer cells and immortalized with oncogenic viruses (e.g., Epstein Barr Virus). Exemplary cell lines which can be used in this aspect are described in U.S. Provisional Application Serial No. No. 60/236,549, filed Sep. 29, 2000, the entirety of which is incorporated herein by reference.

Samples can be homogeneous, comprising a single cell type (e.g., as in a small format or ultrasmall format microarray), or can be heterogeneous, comprising at least one additional type of cell or cellular material in addition to abnormally proliferating cells (e.g., as in large format microarrays where samples are generally larger than 0.6 mm in diameter). For example, the sample can comprise abnormally proliferating cells and at least one of: fibrous tissue, inflammatory tissue, necrotic cells, apoptotic cells, normal cells, and the like.

In another aspect, one or more tissue microarrays are provided comprising tissue samples which fail to express, or express an abnormal level or for, of one or more pathway molecules. For example, in one aspect, one or more tissue microarrays are provided which fail to express, or express an abnormal level or form of a biomolecule which is part of a GPCR pathway, such as a GPCR and/or its cognate ligand.

In one aspect, the microarray 13 comprises tissue and/or cell samples from one or more patients which have been exposed to a drug or agent or environmental condition. The patient may have one or more underlying and/or concurrent diseases or pathological conditions. In one aspect, samples are obtained from a plurality of patients who have been exposed to different levels of a drug or agent, while in another aspect, tissue samples are obtained from patients who have been exposed for varying periods of time to a drug or agent or environmental condition.

Although in a preferred aspect of the invention, the microarrays 13 comprise human specimens, in one aspect of the invention, specimens from other organisms are arrayed. In one aspect, the microarray 13 comprises tissues from non-human animals which provide a model of a disease or other pathological condition. Such animals can be genetically engineered or can be recombinant inbred strains (e.g., such as mice). In one aspect, a microarray 13 is provided comprising tissues from non-human animals expressing different doses of the same cell proliferation gene or tumor suppressor gene. Non-human animals encompassed within the scope of the invention include, but are not limited to mice, rats, swine, dogs, rabbits, primates, and the like. Methods for generating these animals are known in the art.

In one aspect, tissues are obtained from animals which have either spontaneously developed cancer or who have received transplants of tumor cells. In another aspect of the invention, the microarray 13 comprises tissues from non-human animals which have spontaneously developed cancer or who have received transplants of tumor cells, and which have been treated with a cancer therapy.

In still other aspects, tissues from animals exhibiting an aberrant immune response are arrayed. The response may be part of a chronic condition (e.g., in an animal model of Crohn's disease or asthma) or part of an acute response (e.g., a response to LPS). When the array represents tissues from an animal model having a chronic disease, the array can further include tissues representing different stages of the disease, e.g., such as a remission period or an exacerbation period.

The microarray 13 can additionally, or alternatively, comprise tissues from a non-human animal having the disease or condition which has been exposed to a therapy for treating the disease or condition (e.g., drugs, antibodies, protein therapies, gene therapies, antisense therapies, combinations thereof, and the like). In some aspects, the non-human animals can comprise at least one cell containing an exogenous nucleic acid (e.g., the animals can be transgenic animals, chimeric animals, knockout or knockin animals). Preferably, arrays from non-human animals comprise multiple tissues/cell types from such a non-human animal. In one aspect, tissues/cells at different stages of development are arrayed.

Construction of Tissue Microarrays

Tissue microarrays 13 are generated by obtaining donor tissues from any of the tissue sources described above, embedding these tissues, and obtaining portions of the embedded tissue for placement in a “recipient block,” a block of embedding matrix which can subsequently be sectioned, each section being placed on any of the substrates described above. Therefore, in one aspect, the invention encompasses recipient blocks for forming any of the microarrays 13 disclosed above.

Embedding Tissues: Forming Donor Blocks

In one aspect of the invention, tissues are obtained and either paraffin-embedded, plastic-embedded, or frozen. When paraffin-embedded tissues are used, a variety of tissue fixation techniques can be used. Methods of fixing tissues and identifying appropriate targets in a donor block are described in U.S. Patent Application Serial No. 60/234,493, filed Sep. 22, 2000, the entirety of which is incorporated by reference herein.

Donor blocks also can be generated which comprise cells rather than tissues. For example, the donor blocks can comprise embedded cells obtained from cell suspensions. Cells used to form the donor blocks can be obtained from cell culture (e.g., from primary cell lines or continuous cells lines), from dissections, from surgical procedures, biopsies, pathology waste samples (e.g., by mincing or otherwise disassociating tissues from these samples), as well as from bodily fluids (e.g., such as blood, plasma, sera, leukophoresis samples, and the like). Cells can also be obtained after one or more purification steps to isolate cells of a particular type (e.g., by dissection, flow sorting, density gradient centrifugation, and the like).

Cells are preferably washed one or more times in a suitable buffer which does not lyse the cell and are collected by centrifugation. After removing substantially all of the buffer, cells are resuspended gently in a volume of embedding material and transferred in the embedding material to a mold, such as a support web or plastic block, for hardening or freezing in the case of a cryogenic matrix. After the mold is removed, at least one section from the block should be evaluated to verify sample integrity (e.g., to validate the presence of suitable numbers of cells with acceptable morphology and/or to determine that cells express or fail to express one or more biomolecules). Cell donor blocks should comprise at least about one cell and preferably comprise at least about 50, at least about 10², at least about 10³, at least about 10⁴, at least about 10⁵, at least about 10⁶, at least about 10⁷, and at least about 10⁸ cells.

Forming the Recipient Block

In one aspect, microarrays according to the invention are constructed by coring holes in a recipient block comprising an embedding substance (e.g., paraffin, plastic, or a cryogenic media) and placing a tissue sample from a donor block in a selected hole. Holes can be of any shape and size, but are preferably made in a regular pattern. In one aspect of the invention, the hole for receiving the tissue sample is elongated in shape. In another aspect, the hole is cylindrical in shape.

While the order of the donor tissues in the recipient block is not critical, in some aspects, donor tissue samples are spatially organized. For example, in one aspect, donor tissues represent different stages of disease, such as cancer, and are ordered from least progressive to most progressive (e.g., associated with the lowest survival rates). In another aspect, tissue samples within a microarray 13 will be ordered into groups which represent the patients from which the tissues are derived. For example, in one aspect, the groupings are based on multiple patient parameters that can be reproducibly defined from the development of molecular disease profiles. In another aspect, tissues are coded by genotype and/or phenotype.

For example, tissue samples may be arrayed in order of their progression through the cell cycle by obtaining a sample of a tissue core and determining what stage of the cell cycle it is in by virtue of the expression of particular biomolecules and/or cytological criteria. The tissue core is then placed in a known location in a recipient block and additional tissue cores are obtained which represent different stages of the cell cycle. Duplicate cores can also be provided. A section of the recipient block is obtained to verify that tissue cores within the block are at the stage of the cell cycle identified, and the block is then used to generate a plurality of microarrays representing different stages of the cell cycle.

In some aspects, tissue samples are obtained which fail to express or which express altered levels or forms of a GPCR pathway molecule. For example, recipient blocks can be generated by obtaining tissue samples from tissues which fail to express early, middle and late pathway genes. As used herein, “early pathway genes” are genes whose expression effects the expression of multiple downstream genes (at least about 5), such that perturbing the expression of these genes will effect multiple genes in the pathway. “Middle pathway genes” are genes whose expression is required for the expression of at least about 2 but less than five downstream genes, while “late genes” are those which are downstream in the pathway and whose expression effects only one or a few (e.g., less than about 2 pathway molecules). Recipient blocks comprising tissues having defects in the expression of early, middle and late pathway genes can be generated by obtaining tissue sections of an embedded tissue sample (e.g., a donor block), and subsequently coring the tissue sample if it produces the desired pattern of expression. Recipient blocks are validated by obtaining representative section(s) of the block and reacting the sections with a plurality of molecular probes which can react with early, middle, and late pathway genes and their products (which may include the expression products of other genes or various metabolites or cellular constituents.

Tissue samples on the microarray 13 can be arranged according to expression of biomolecules, if this is known, or by characteristics of the tissue source, including exposure of the tissue source to particular treatment approaches, treatment outcome, or prognosis, or according to any other scheme that facilitates the subsequent analysis of the samples and the data associated with them.

The recipient block can be prepared while tissue samples are being obtained from the donor block. However, in one aspect, the recipient block is prepared prior to obtaining samples from the donor block, for example, by placing a fast-freezing, cryo-embedding matrix in a container and freezing the matrix so as to create a solid, frozen block. The embedding matrix can be frozen using a tissue freezing aerosol such as tetrafluorethane 2.2 or by any other methods known in the art. The holes for holding tissue samples can be produced by punching holes of substantially the same dimensions into the recipient block as those of the donor frozen tissue samples and discarding the extra embedding matrix.

Information regarding the coordinates of the hole into which a tissue sample is placed and the identity of the tissue sample at that hole is recorded, effectively addressing each sublocation 13s on the microarray 13. In one aspect of the invention, data relating to any, or all of, tissue type, stage of development or disease, individual of origin, patient history, family history, diagnosis, prognosis, medication, morphology, concurrent illnesses, expression of molecular characteristics (e.g., markers), and the like, is recorded and stored in a database, indexed according to the location of the tissue on the microarray 13. Data can be recorded at the same time that the microarray 13 is formed, or prior to, or after, formation of the microarray 13.

The coring process can be automated using core needles coupled to a motor or some other source of electrical or mechanical power. Methods for automating tissue arraying are described in U.S. Pat. No. 6,103,518, in International Applications WO 99/44062 and WO 99/44062, in U.S. patent application Ser. No. 09/779,753, entitled “Frozen Tissue Microarrayer,” filed Feb. 8, 2001, and in U.S. patent application Ser. No. 09/779,187, entitled “Stylet For Use With Tissue Microarrayer and Molds,” filed Feb. 8, 2001, the entireties of which are incorporated by reference herein.

In one aspect of the invention, large formats microarrays 13 are provided which comprise at least one sublocation greater in at least one diameter than about 0.6 mm., about 1.2 mm or about 3.0 mm. In another aspect, at least one sublocation comprises a heterogeneously expressed biomolecule which is expressed in less than about 80% of cells in a given tissue type and which is diagnostic of a disease. In a further aspect of the invention, the large format microarray 13 comprises at least one sublocation 13s comprising at least two different cell types or cellular material (e.g., any of abnormally proliferating cells (e.g., cancerous cells), stromal cells, extracellular matrix, necrotic cells and apoptotic cells).

Large format microarrays 13 can be used alone or in conjunction with small format microarrays 13 (microarrays 13 in which individual sublocations 13s are less than 0.6 mm in diameter). In one aspect of the invention, a large format microarray 13 is used in conjunction with a small format microarray 13 derived from the same patient's tissue sample. In this aspect, the large format microarray 13 can be used to demonstrate that the biological characteristics of the smaller sublocations of the small format microarray 13 are representative of the biological characteristics within a larger sample. Methods of constructing large format microarrays 13 are disclosed in U.S. patent application Ser. No. 09/780,982, filed Feb. 8, 2001, entitled, “Large Format Microarrays”, the entirety of which is incorporated by reference herein. In some applications, such as where a limiting amount of sample is available to be analyzed, an ultrasmall format microarray is generated comprising at least one tissue sample 0.3 mm or smaller. Microarrays comprising tissue samples of varying sizes can also be provided (i.e., including at least two of any of large format, small format, and ultrasmall format tissue samples). Preferably, different sizes of tissue from the same tissue block are provided. Such microarrays can be used to validate that biomolecules detected in a large format microarray will also be detectable in a small format or ultrasmall format microarray.

Tissue Information System for Evaluating GPCR-Pathway Mediated Physiological Responses

The invention provides a tissue information system 1 (shown in FIG. 3) for evaluating physiological responses mediated by alterations in the expression of GPCR pathway molecules. The system 1 enables a user to access, organize, and display information relating to tissue microarrays 13. In particular, the system provides a specimen-linked database enabling a user to evaluate the physiological responses of organisms whose tissues are included in the arrays. The tissue information system 1 comprises at least one user device 3 connected to a network 2. In one aspect, the network is wide area network (WAN) to which the at least one user device 3 is directly connected. However, in another aspect, user device 3 is connected to a WAN indirectly through a local area network (e.g., via a proxy server).

Because the user device 3 is connected to the network 2, individual steps of accessing, organizing, and displaying can be performed on one, or a plurality, of user devices 3 at different physical locations. Thus, in one aspect of the invention, one or more tissue microarrays are each screened at physically distant locations, for example, in different laboratories, hospitals, or companies, and the information obtained from the microarrays screened at each location is correlated with tissue information included within the specimen-linked database 5. Multiple users can both access and add to information within the database 5.

Accessing the system 1 through the user device 3 results in an interface 6 being displayed on a display of the device 3. The interface 6 comprises at least one link to a specimen-linked database 5 which comprises tissue information. In one aspect, the database 5 is also coupled to an information management system (IMS) 7 which comprises both information search functions and relationship determination functions for presenting information to the user in a useable form.

The device 3 comprises a processor and further includes processor readable storage media or electronic memory that can be accessed by the processor. Processor media includes volatile and nonvolatile media, such as RAM, ROM, EPROM, flash memory, CD-ROM, digital versatile disks (DVD), optical storage media, cassettes, tape, discs, and the like. The device 3 can further include multimedia rendering functions by including audio and video components (not shown). In one aspect, the device 3 also comprises an operating system (e.g., such as Microsoft Windows, UNIX X-Windows, or Apple Macintosh System) and one or more application programs, including an Internet or Web browser, such as Microsoft's Internet Explorer™, or Netscape® (see, as described in Internet Starter Kit by Adam Engst, Corwin Low and Michael Simon, Second Edition, Hayden Books, 1995, the entirety of which is incorporated by reference herein).

Web browsers enable a user of the user device 3 to click on portions of an interface 6 displayed on the display of a user device 3, triggering a response by the system 1. In one aspect, the response by the system 1 is to download and display tissue information on the interface 6 or to provide links to sources of tissue information. In addition to browsers, other networking systems can be included in the tissue information system 1, such as routers, peer devices, common network nodes, modems, and the like.

Suitable devices 3 connectable to the network 2 which are encompassed within the scope of the invention, include, but are not limited to, computers, laptops, microprocessors, workstations, personal digital assistants (e.g., palm pilots), mainframes, wireless devices, and combinations thereof. In one aspect, the device 3 comprises a text input element 8, such as a key board or touch pad, enabling the user to input information into the system 1. In another aspect, navigating devices 20 are coupled to the device 3 to allow the user to navigate an interface 6. Navigating devices 20 include, but are not limited to, a mouse, light pen, track ball, joystick(s) or other pointing device.

In one aspect, the system 1 comprises at least one server 4. The server 4 provides access to one or more data storage media such as hard disks or hard disk arrays. In one aspect, the server 4 maintains the database 5 on one of these hard disks. In one aspect, the server 4 comprises one or more applications, including the IMS 7, which permits a user to access information within the database 5, as well as to implement programs for determining relationships between data in the database 5 and tissues on the microarray 13. In another aspect, another application program is provided which implements the search function of the IMS 7. In a further aspect, application programs which retrieve records also perform user-defined operations on the records (e.g., such as creating folders in which to store records of particular interest to a user). Applications programs ordinarily are written in a general purpose host programming language, such as C<++>; however, also include user-defined statements written in a relational query language such as SQL. In some aspects, a web application is provided which includes executable code necessary for the generation of SGL statements. The application can include configuration files which include pointers and addresses to the various software applications included within the server as well as to external and internal databases that must be accessed to service user requests.

In further aspects of the invention, the system 1 comprises information output modules 30 (e.g., printers) for outputting and reporting information from the database 5. The system can also comprise information input modules 31 (e.g., scanners), for receiving information from a user, such as scanned data.

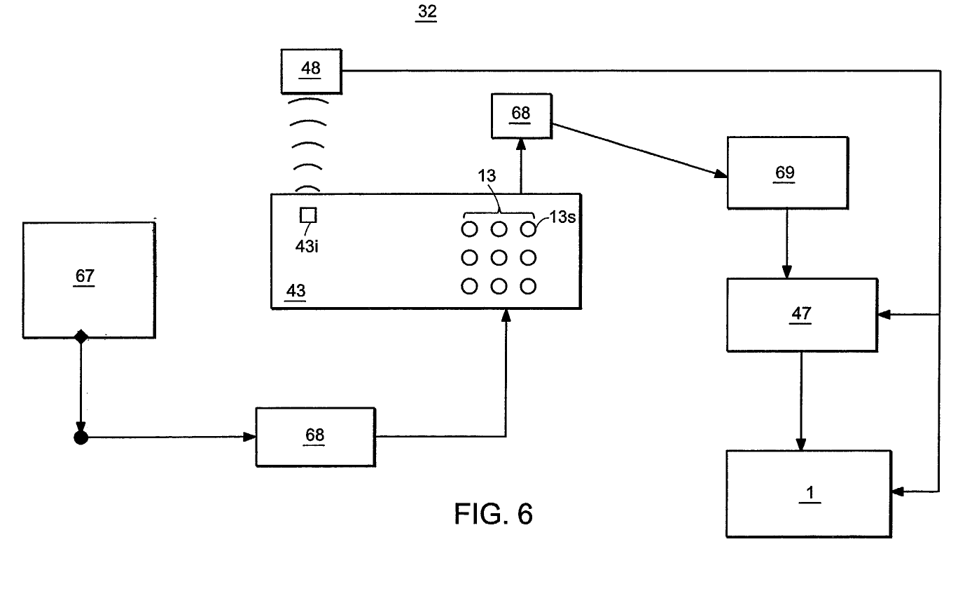

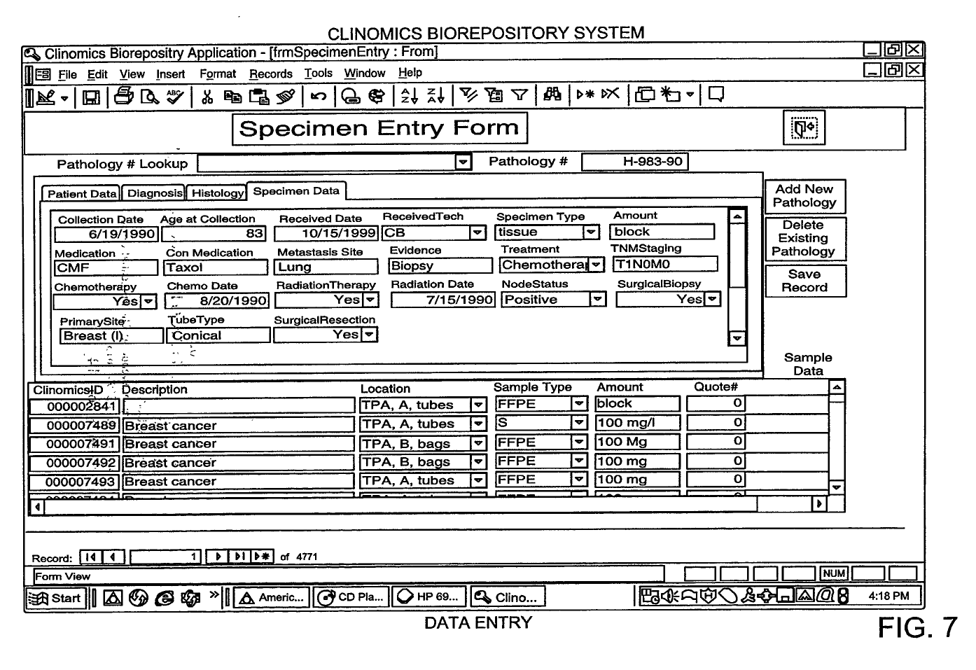

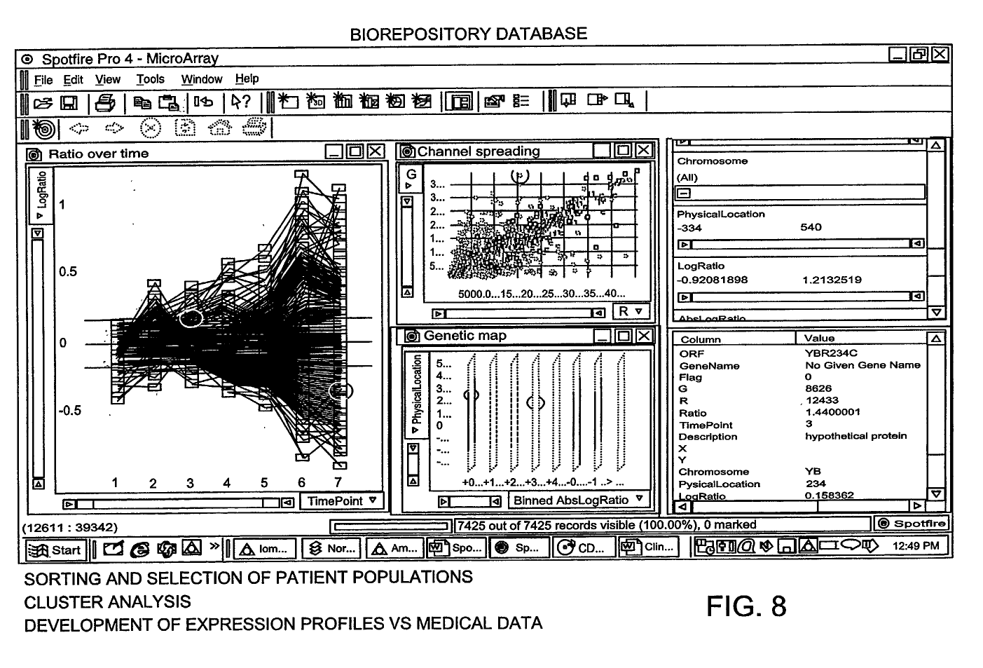

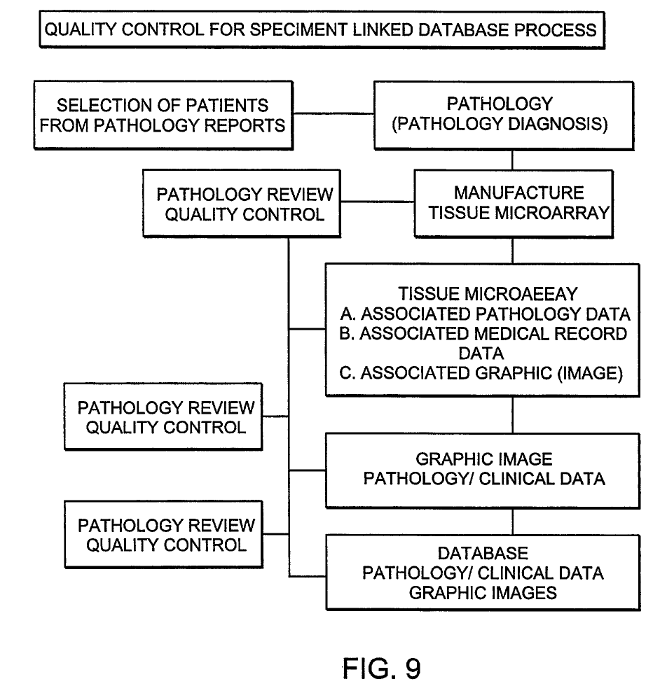

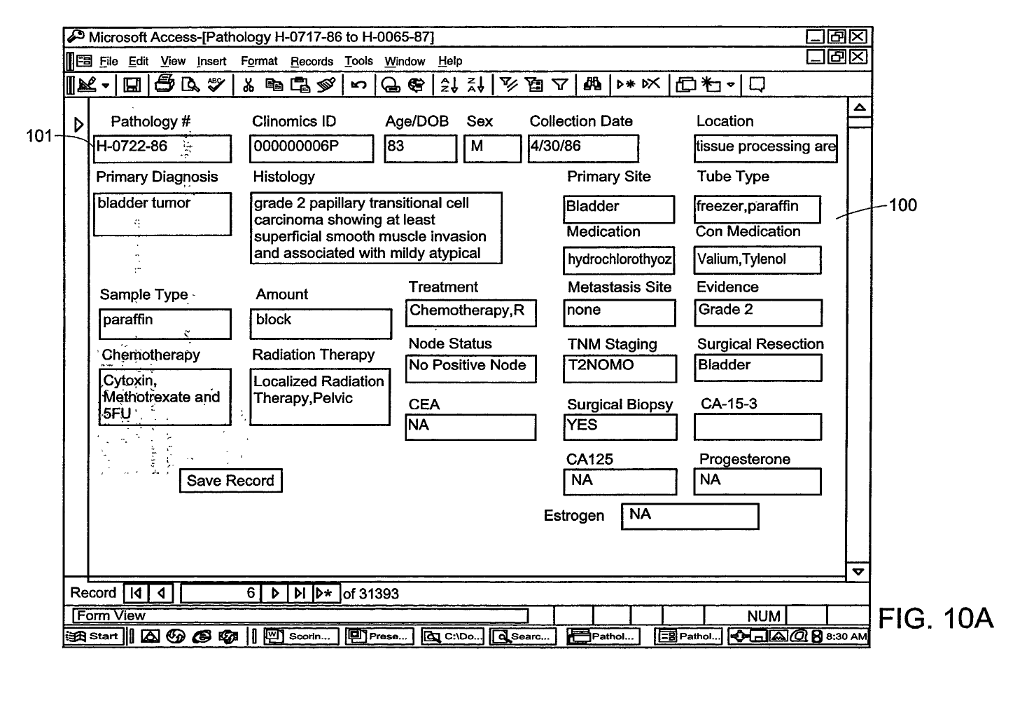

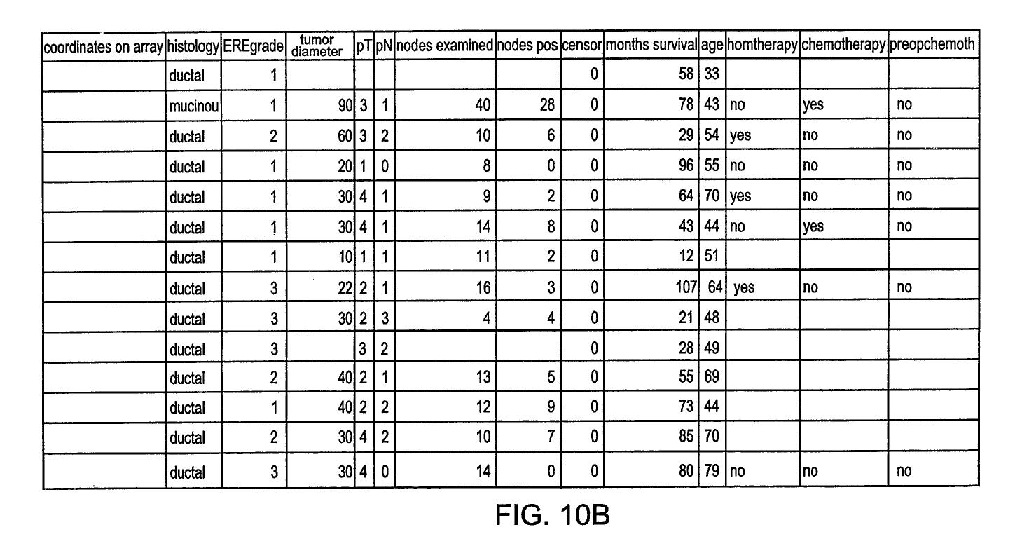

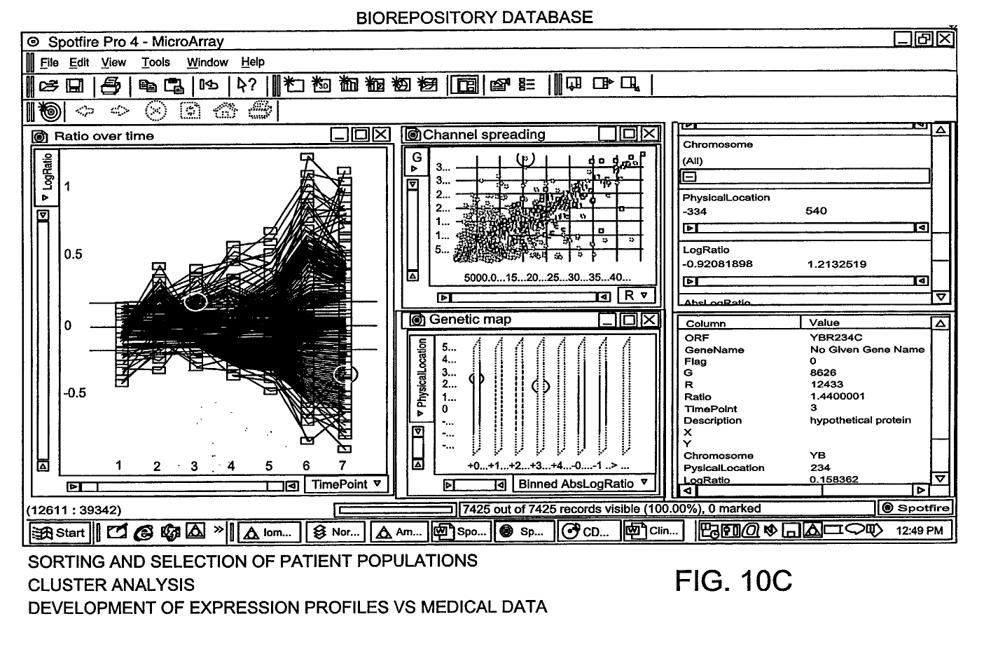

In still another aspect of the invention, a molecular profiling system 32 (such as the one shown in FIG. 6) is provided which is connectable to the device 3. In one aspect, molecular profiling data is automatically inputted into the database 5, and a user accessing the system 1 has immediate access to this data.

Specimen-Linked Database

Information within the specimen-linked database 5 is dynamic, being added to and refined as additional users access the database 5 through the system 1. In one aspect, inputted information at least comprises information relating to the analyses of the tissue microarrays 13 described above and the database 5 organizes this information according to a data model. Data models are known in the art and include flat file models, indexed file models, network data models, hierarchical data models, and relational data models. Flat file models store data in records composed of fields and are dependent upon the particular applications comprising the IMS 7, e.g., if the flat file design is changed, the applications comprising the IMS 7 must also be modified. Indexed file systems comprise fixed-length records composed of data fields and indexes which group data fields according to categories. Spreadsheets and text files can also be used.

A network data model also comprises fixed-length records composed of data fields which are indexed according to categories. However, network data models provide record identifiers and link fields to connect records together for faster access. Network data models further comprise pointer structures which provides a shorthand means of identifying linked records. Hierarchical data models comprise fixed-length records composed of data fields, indexes, record identifiers, link fields, and pointer structures, but further represent the relationship of different records in a database in a tree structure. Hierarchical data models are described further in U.S. Pat. No. 5,980,096, the entirety of which is incorporated by reference herein.

In contrast, relational data models comprise tables comprising columns and rows of data elements or attributes. Attributes provide information about the different facts stored within the database 5. Columns within the table comprise attributes of the same data type (e.g., in one aspect, all information relating to patient X's drug exposure), while each row of the table represents a different relationship (e.g., row one, representing dosage, row two representing efficacy, row three representing safety). As with network data models, and hierarchical data models, relational database models link related information within the database.

Any of the data models described above can be used to organize information within the database 5 into information categories to facilitate access by a user of the tissue information system 1. In a preferred aspect, a system operator, i.e., the user who provides access to the tissue information system to other users, determines the parameters which define a particular information category recognized by a particular data model.

For example, in one aspect, the system operator determines the fields that are used to define the information category “drug exposure.” In this aspect, the system operator may determine that these fields should include: “types of drugs to which the patient was exposed”; “frequency of exposure”; “dose at each exposure”; “physiological response to exposure”; “tests used to measure physiological responses”; “molecular response to exposure”; “tests used to measure molecular responses”; and the like. Similarly, the system operator may determine that fields which define the information category “medical history of a patient” should encompass all information obtained by health care workers at any time during the patient's life, as well as information relating to tests performed by health care workers, or should encompass only selected portions of such records. It should be obvious to those of skill in the art that information categories determined by the system operator can overlap in the types of information contained within them. For example, information relating to medical history could include information relating to a patient's drug exposure. In one aspect, therefore, the database 5 further comprises links between different information categories which comprise areas of overlap.

The parameters defined by the system user are included within a database dictionary portion of the database 5 and in one aspect, a user other than the system operator can access the database dictionary on a read-only basis to determine what parameters were used to define a particular information category. In another aspect of the invention, a user of the system can request that additional parameters be included in the definition of an information category, and, subject to the approval of the system operator, the definition of the information category can be modified as the database expands. In a further aspect, the database 5, for example, as part of the dictionary can include a table comprising word equivalents to facilitate searching by the IMS-7. In some aspects, the table comprises codes representing community accepted definitions of diagnoses, anatomic locations and the like (e.g., such as SNOWMED codes, DSM-IV-TR codes) or accepted genetic nomenclature (e.g., UNIGENE codes).

In one aspect, new information inputted into the system 1 is stored within a temporary database and is subject to validation by the system operator prior to its inclusion in the portion of the database 5 to which all users of the system have access to.

In another aspect, data within the temporary database, is fully able to be accessed and compared to information within the specimen-linked database 5; however, users of the system 1 are alerted to the fact that data within the temporary database has not necessarily been validated (e.g., repeated or evaluated as to quality). In this aspect, the information categories included within the temporary database can include information relating to the time and date on which the new information was inputted into the system 1.

In one aspect of the invention, information within information categories is derived from an analysis of any of the tissue microarrays described above. For example, in one aspect, the database 5 comprises information reflective of “whole body microarrays” which have been evaluated by user(s). In this aspect, information included within the database encompasses information relating to the types of tissue on the microarray and relating to biological characteristics of the tissue source (e.g., such as patient information). In another aspect, the database 5 comprises information including, but not limited to, the sex and age of the tissue source, underlying diseases affecting the tissue source, the types of drugs or other therapeutic agents being taken by the tissue source, the localization of the drugs and agents in the different tissues of the microarray, and the effects of the drugs and agents on the different tissues of the microarray, environmental conditions to which the tissue source has been, and is being exposed to, as well as the lifestyle of the tissue source (e.g., moderate or no exercise, alcohol, tobacco consumption, and the like), cause of death and age of death (if appropriate).

In further aspects of the invention, information from a plurality of microarrays 13 is used to create the database 5, providing information relating to populations of individuals (e.g., such as demographic and/or epidemiological information). In one aspect, information relating to microarray(s) 13 comprising at least one disease tissue sample (e.g., a tissue sample expressing biological characteristics associated with disease) is included within the database 5. In one aspect, this information relates to biological characteristics which define different stages of the disease (e.g., biological characteristics which are associated with different stages of cancer). In another aspect, information relating to the biological characteristics of normal tissues from the same or different patients is also included within the database 5. In a further aspect, patient information relating to the tissue sources of tissues at different sublocations 5 on microarray(s) 13 is included within the database, providing information such as gender, age, underlying diseases, family information, cause and time of death if appropriate, information relating to treatment with drugs or other therapeutic agents (e.g., such as protein or nucleic acid-based therapeutic agents), and/or exposure to chemotherapy, radiotherapy, surgery, environmental conditions, and the like.

While in one aspect, the database 5 comprises information relating to human tissues, in another aspect, the database 5 also includes information from non-human tissues (e.g., animals, plants, and/or genetically engineered animals or plants). For example, in one aspect, the database 5 includes information relating to the biological characteristics of non-human tissues which have been exposed to any of drugs, antibodies, protein therapies, gene therapies, antisense therapies, and the like. In some aspects, the biological characteristics of tissues from non-human individuals which have been genetically engineered to overexpress or underexpress desired genes are included within the database 5. In a further aspect, information within the database 5 also includes information from cell lines (normal and/or cancer cell lines) which have been genetically engineered to express desired genes (e.g., cell proliferation genes or tumor suppressor genes or modified forms of such genes).

In one aspect, the database comprises information relating to tissues from different recombinant inbred strains of individuals (e.g., mice). Such information includes, but is not limited to, the allele carried at one or more loci, haplotype information, and information relating to the expression of one or more proteins encoded by these loci. In a further aspect, information relating to diseases associated with particular alleles or haplotypes are further included within the database.

In one aspect, the database 5 comprises molecular profiling data relating to the expression of one or more GPCR pathway biomolecules. In one aspect, molecular profiling data is obtained from any of normal tissue, diseased tissue (including tissues at different stages of disease), different developmental stages from one or more different types of organisms, and from tissues which have been genetically engineered to include different doses or altered forms of gene(s). Molecular profiling data from whole body microarrays as well as microarrays reflecting populations of individuals can also be included within the database 5. In one aspect, molecular profiling data includes the expression pattern of a plurality of GPCR pathway genes expressed during cancer, or in a patient having one or more of an autoimmune disease or other pathological immune response, a neurodegenerative disease (either chronic or acute), a neuropsychiatric disorder, a respiratory disorder, a skin disorder, a gastrointestinal disorder, a cardiovascular disorder, an endocrine disorder, and the like. In another aspect, molecular profiling data includes data relating to genes expressed during selected physiological processes (e.g., such as tissue responses to ischemia).

While in one aspect, information within the database 5 is obtained from tissues provided on the microarrays 13 described above, tissue information can also be obtained from a variety of other sources, such as test samples assayed alongside the tissue microarrays 13 (e.g., using profile array substrates) or test samples which have been assayed independently of tissue microarrays 13, or tissue samples from cell lines, or tissue panels from living patients or from archived tissues, and the like. Information relating to nucleic acid microarrays, protein, polypeptide, peptide, and other biomolecule arrays can also be included within the database, irrespective of whether information from a corresponding tissue microarray 13 has also been obtained. As used herein, although the database is described as being “specimen-linked,” the database can also include data unrelated to specific test specimens.

In one aspect, the specimen linked database 5 can be organized to facilitate information retrieval by the IMS 7 by providing a plurality of “subdatabases”, each of which comprises information relating to a particular category of tissue information. For example, in one aspect, the subdatabases comprise information relating to any of: oncology, cardiovascular diseases, respiratory diseases, renal diseases, gastrointestinal diseases, liver diseases, metabolic diseases, endocrine diseases, infectious diseases, inflammatory diseases, musculoskeletal diseases, neurological diseases (including neurodegenerative and neuropsychiatric diseases), dermatological diseases, gynecological diseases, and urological diseases. Preferably, each of these subdatabases includes records comprising information relating to the expression of GPCR pathway molecules in tissues from patients having these diseases.

In another aspect, subdatabases are restricted to particular types of information and include, but are not limited to, sequence subdatabases, protein structure subdatabases, chemical formula/structure subdatabases, expression pattern subdatabases (e.g., providing information relating to the expression of genes in different tissues), information relating to drug targets and drug leads (e.g., including, but not limited to information relating to compound toxicity, side effects, efficacy, metabolism, drug interactions), as well as literature subdatabases, medical history subdatabases, demographic information subdatabases, and the like.

In one aspect of the invention, data within the database 5 is defined using SNOMED® Clinical Terms™. For example, different clinical concepts (e.g., cardiovascular disease, neurodegenerative disease, autoimmune disease, cancer, reproductive disease, neuropsychiatric diseases) are assigned unique concept identifiers which are represented within a “Concept Table” within the database 5. Concepts can be defined by codes, such that a string of codes can be used to cross reference data from a plurality of databases and subdatabases.

In a further aspect, the database 5 stores uncompressed raw data files, such as for example, microscopy and histological data obtained from the tissues. In this aspect, the database 5 is of a magnitude which enables storage of memory intensive files, and the network 2 connection enables high speed (T-1, T-3 or higher) transmission of the data to the user. In still another aspect of the invention, data relating to an image of the test tissue is stored within the database 5 and the image can be displayed by the user upon accessing the database 5.

Thus, as described above, the specimen-linked database 5 according to the invention makes information available concurrently from a number of different sources to enable a user to practice “genomic medicine,” i.e., to develop diagnostic and treatment modalities based not only on the physiological responses of a patient, but also on the biomolecular responses of a patient. As illustrated in the table below, in one aspect, a genomic medicine database is provided which comprises a plurality of subdatabases, including, but not limited to, a patient information subdatabase, a medical information subdatabase, a pathology information subdatabase, and a genomic information subdatabase. Preferably, the genomic information database comprises information about a plurality of GPCR pathway biomolecules.

As can be seen from the table, information in one database may overlap (i.e., be repeated) in another database. For example, a pathology subdatabase can included molecular information relating to a particular disease, just as can a genomics database, and may also include additional information, such as information identifying the correlation between a particular marker and a morphological characteristic.

Genomic Medicine Database

| | | Pathology | |
|---|---|---|---|
| Patient Information | Medical Information | Information | Genomic Information |
| Subdatabase | Subdatabase | Subdatabase | Subdatabase |
| Demographics | Diagnosis | Diagnosis | DNA |
| Life style | Other conditions | Histology | Protein |
| Epidemiology | Concurrent Illness | Clinical Data | mRNA |
| Family History | Medications | Molecular Markers | |
| | Outcome Survival | | |

Physiological Response Database

In a preferred aspect of the invention, the database 5 comprises information relating to the physiological responses of patients to particular conditions, such as diseases, pathological conditions, drugs or agents, environmental conditions, and the like. Physiological responses include, but are not limited to, cellular metabolism, energy metabolism, nucleic acid metabolism, signal transduction, progression through the cell cycle, cell transformation, DNA repair, secretion, subcellular localization and processing of cellular constituents (e.g., including RNA splicing, protein modification and cleavage), cell-cell interactions, cell migration, cell adhesion, growth, differentiation, apoptosis, immune responses, neurotransmission, ion transport, sugar transport, lipid metabolism, and the like. The database 5 also can include information relating to kinetic parameters which govern physiological responses. For example, the database can include information relating to dissociation constants, Michaelis Menton constants, inhibition constants, catalytic constants, circulating half-life, excretion rates, and the like.

In one aspect, physiological responses are evaluated by monitoring the expression of a plurality of biomolecules representing at least one GPCR pathway in a tissue sample (“GPCR pathway biomolecules”) and using the database 5 to identify correlations between an expression pattern observed and the likelihood that the source of the tissue sample has been exposed to one or more conditions. Preferably, physiological responses are evaluated by monitoring the expression of GPCR pathway biomolecules in a plurality of tissues, and more preferably, in whole body microarrays representing different populations of patients which share one ore more traits.

In one aspect, the database 5 comprises records relating to biomolecules which are expressed or inhibited upon activation of a particular GPCR pathway biomolecules. For example, the database can include expression information relating to any one or more of a serotonin receptor (e.g., 5-hydroxytryptamine 1A, 1B, 1C, 1D, 1F, 2A, 2C, 5A and/or 5B receptors), an adenosine receptor (e.g., an adenosine A1 receptor, an adenosine A2A, A2B, A3, P2U, and/or P2Y), uridine nucleotide receptor, an adrenergic receptor (e.g., α-1A, 1B, 1C, 2A, 2B, 2C, and/or β-1, 2, and/or 3), angiotensin receptor, bombesin receptor (e.g., bombesin Type 3, Type 4), neuromedin B receptor, gastrin-releasing peptide receptor, bradykin receptor, C5A-anaphylatoxin receptor, a cannabinoid receptor (e.g., Type 1, Type 2, Type A), gastrin receptor, dopamine receptor (e.g., dopamine 1A, 1B, D2, D3, D4), endothelin receptor (e.g., endothelin A, endothelin B), formyl-methionyl peptide receptor, gonadotrophin releasing hormone receptor, glycoprotein hormone receptor, histamine receptor (H1 and/or H2), interleukin-8 receptor (e.g., interleukin 8A and 8B), adrenocorticotrophin receptor, melanocortin receptor, melanocyte stimulating hormone receptor, muscarinic receptor (e.g., M1, M2, M3, M4, M5 receptors) neurokinin receptor, olfactory receptor, opiod receptor (delta, kappa, mu, and/or X receptors), opsin (blue or red/green sensitive), such as a rhodopsin receptor, parathyroid hormone receptor, secretin receptor, vasoactive intestinal peptide receptor, extracellular calcium-sensing receptor, metabotropic glutamate receptor, prostanoid receptor (EP1, EP2, EP3, EP4), platelet activating factor receptor, thromboxane receptor, somatostatin receptor (Type 1, 2, 3, and/or 4), Burkitts' Lymphoma receptor, EB1I orphan receptor, EDG1 orphan receptor, G10D orphan receptor, GPR3 orphan receptor, GPR6 orphan receptor, GPR10 orphan receptor, LCR1 orphan receptor, mas oncogene, RDC1 orphan receptor, SENR orphan receptor, calcitonin receptor, parathyroid hormone receptor, secretin receptor, extracellular calcium sensing receptor, a GABA receptor, HF1AO41, HOFNH30, HCEGH45, HPRAJ70, HGBER32, HFIZO41, HIBCD07, a GPR receptor, including, but not limited to, GPR1, GPR 27, GPR30, CPR31, GPR34, GPR 35, GPR37, GPR45, GPR52, GPR55, GPR61, GPR62, GPR63, GPR77, GPR88, epidermal growth factor (EGF)-TM7 protein, Ca(2+)(o)-sensing receptor (CaR), a leucine-rich repeat-containing G protein-coupled receptor, chemokine receptor, pheromone receptor, r, tachykinin receptor, melanocortin receptor, a viral GPCR receptor, VPAC(1), VPAC(2), PAR1, CRF-R, Emr1, HIBCD07, HLWAR77, an SREB GPCR, an Edg receptor, a lysophospholipid receptor, SALPR, GH-secretagogue receptor (GHS-R), a PACAP receptor, an EBI-2 GPCR, a vasopressin receptor (e.g., V2 vasopressin renal receptor (V2R)) a follicle stimulating hormone receptor, lutropin-chroiogonadotrpic hormone receptors, thyrotropin receptor, Mas proto-oncogene receptor, RDC1, a class E cAMP receptor, ocular albinism protein receptors (e.g., OA1), frizzled receptors, smooth receptors, Mlo receptors, nematode chemoreceptor, unclassified GPCRs, class Y GPCR, homologous, mutated, or variant forms thereof, and any biomolecules whose expression is turned on or off upon activation of these receptors, or whose expression negatively or positively regulates the expression of these receptors, and/or their homologous, mutant or variant forms. Preferably, the database 5 includes information relating to the expression of at least 10 of these receptors, at least 20 of these receptors, at least 50 of these receptors, or all of these receptors in a plurality of different tissues (e.g., such as the whole body microarrays described above). More preferably, the database 5 includes information relating to the expression of phosphorylated and unphosphorylated forms of these receptors.

In other aspects, information relating to GPCRs can be related to the expression of other pathway molecules to determine interrelationships between multiple molecular pathways. For example, in one aspect, the expression of at least on GPCR pathway molecule is related to the expression of one or more the cell cycle pathway molecules. For example, in addition to information relating to the expression of the GPCR pathway molecule, the database can comprise information relating to the expression of one or more of SL1, C42, cdk1, cdk7, CycH, C42, C14, PCNA, R11, R10, CycD, p21, S9, CycA, RPA, S9, CycB, p68, primase, R2, Polα, CycE, Skp1, CBF3, C26, E2f, DMP1, cdc25a, CycD, cdk4/6, Gadd45, p26, p27, p53, p57, C17, C18, C23, C21, C13, C28, C30, C37, C38, C39, E20, pS76, Chk1, C-TAK1, APC, cdc25C, cdk1, cks1, Wee1, Myt1, Plk1, C15, C41, C37, C6, pTY4Y15, pT161, pS216, pY15, and other molecules in the cyclin-E2F cell cycle control system (see, e.g., as described at http://discover.nci.nih.gov/kohnk/interaction_maps.html), and homologs, mutants and/or variants thereof.

In another aspect, the physiological response database 5 also comprises information relating the expression of one or more DNA repair genes. For example, the database can comprise information relating to the expression of one or more of Rpase II, TBP, TAF_{II}250, P36, RHA, MDM2, p53, p27, CSB, XPB/D, p36, cdk7, cycH, C43, P11, A5, C43, c-Ab1, H7, p16, cycD, cdk4, primase, R2, p21, cycE, cycA, cdk2, PCNA, Polα, p70, N10, N7, S1, S2, S7, S8, S10, S11, S12, S13, S14, S16, S17, p34, rad52, SBF3, Skp1, Skp2, R1, DNAP α, p68, RF-C, FEN-1, ligase 1, Gadd45, XPC, cycD, PARP, karp, Ku80, Ku70, RPA2, HMG, histones, ATM, paxillin, Crk, pRb, RAD51, ss or ds DNA breaks, XPF, XPC, XPA, XPG, DNAPβ, ligaseII, ERCC1, U-glycosylase, BRCA1, pKCα/β, PARP, glycohydrolase, and other genes involved in the p53-MDM2 DNA repair pathway, and homologs, mutants and/or variants thereof.

The physiological response database 5 can also comprise information relating the expression of one or more biomolecules involved in cholesterol metabolism, such as LDL, LDL-receptor, VLDL, HDL, cholesterol acyltransferase, apoprotein E, Cholesteryl esters, ApoA-I and A-II, HMGCoA reductase, cholesterol, and homologs, mutants and/or variants thereof.

In another aspect, the physiological response database 5 can also comprise information relating the expression of one or more biomolecules involved in apoptosis, such as Bcl, Bak, ICE proteases, Ich-1, CrmA, CPP32, APO-1/Fas, DR3, FADD containing proteins, perforin, p55 tumor necrosis factor (TNF) receptor, NAIP. IAP, TRADD-TRAF2 and TRADD-FADD, TNF, D4-GDI, NF-kB, CPP32/apopain, CD40, IRF-1, p53, apoptin, and homologs, mutants and/or variants thereof.

The physiological response database 5 can also comprise information relating the expression of one or more biomolecules involved in blood clotting, such as thrombin, fibrinogen, factor V, Factor VIII-FVa, FVIIIa, Factor XI, Factor Xia, Factors IX and X, thrombin receptor, thrombomodulin (TM), protein C (PC) to activated protein C (aPC). aPC, plasminogen activator inhibitor-1 (PAI-1), tPA (tissue plasminogen activator), and homologs, mutants and/or variants thereof.

In another aspect, the physiological response database 5 also can comprise information relating the expression of one or more biomolecules involved in the flt-3 pathway, such as, flt-3, GRP-2, SHP-2, SHIP, She, and homologs, mutants and/or variants thereof.

In another aspect, the physiological response database also can comprise information relating the expression of one or more biomolecules involved in the JAK/STATS signaling pathway, such as Jak1, Jak2, IL-2, IL-4 and IL-7, Jak3, Ptk-2, Tyk2, EPO, GH, prolactin, IL-3, GM-CSF, G-CSF, IFN gamma, LIF, OSM, IL-12 and IL-6, IFNR-alpha, IFNR-gamma, IL-2R beta, IL-6R, CNTFR, Stat1alpha, Stat1beta, Stats2-6, and homologs, mutants and/or variants thereof.

In another aspect, the physiological response database 5 also comprises information relating the expression of one or more biomolecules involved in a MAP kinase signaling pathway, such as flt-3, ras, raf, Grb2, Erk-1, Erk-2, Src, sos, Shc, Erb2, gp130, MEK-1, MEK-2, hsp 90, JNK, p38, Sin1, Sty1/Spc1, MKK's, MAPKAP kinase-2, JNK/SAPK, and homologs, mutants and/or variants thereof.

The physiological response database 5 also can comprise information relating the expression of one or more biomolecules involved in a PI 3 kinase pathway, such as SHIP, Akt, and homologs, mutants and/or variants thereof.

The physiological response database 5 also can comprise information relating the expression of one or more biomolecules involved in a ras activation pathway, such as p120-Ras GAP, neurofibromin, Gap1, Ral-GDS, Rsbs 1, 2, and 4, Rin1, MEKK-1, and phosphatidylinositol-3-OH kinase (PI-3 kinase), ras, and homologs, mutants and/or variants thereof.

In another aspect, the physiological response database 5 also can comprise information relating the expression of one or more biomolecules involved in an SIP signaling pathway, such as GRB2, SIP, ras, PI 3-kinase, and homologs, mutants and/or variants thereof.

In another aspect, the physiological response database 5 also can comprise information relating the expression of one or more biomolecules involved in an SHC signaling pathway, such as trkA, trkb, NGF, BDNF, NT-4/5, trkc, f NT-3, Shc, PLC gamma 1, PI-3 kinase, SNT, ras, rafi, MEK, MAP kinase, and homologs, mutants and/or variants thereof.

In another aspect, the physiological response database 5 also can comprise information relating the expression of one or more biomolecules involved in a TGF-β signaling pathway, such as BMP, Smad 2, Smad4, activin, TGF-β, and homologs, mutants and/or variants thereof.

In another aspect, the physiological response database 5 also can comprise information relating the expression of one or more biomolecules involved in a T cell receptor based signaling pathway, such as lck, fyn, CD4, CD8, T cell receptor proteins, and homologs, mutants and/or variants thereof.

The physiological response database 5 also can comprise information relating the expression of one or more biomolecules involved in a MHC-1-mediated antigen presentation, such as TAP proteins, LMP 2, LMP 7, gp 96, HSP 90, HSP 70, and homologs, mutants and/or variants thereof.

In a preferred aspect, the physiological response database 5 comprises information relating to the expression of a plurality of pathway molecules in addition to GPCR pathway molecules expressed within whole body tissue microarrays obtained from populations of patients and the database is subdivided to include subdatabases including information relating to specific pathways, such as the ones described above. Additional subdatabases encompassed within the scope of the invention include, but are not limited to, an EGF receptor pathway subdatabases, insulin receptor pathway subdatabases, p53 mediated pathway subdatabases, metabolic pathways subdatabases, HOX gene and other pattern forming gene pathways (e.g., such as hedgehog gene pathways) subdatabases, and the like.

In a preferred aspect, the database also comprises information relating to the expression of one or more tyrosine kinase pathway molecules. Such molecules include, but are not limited to, NTRK1; PTK2; SRK; CTK; TYRO3; BTK; LTK; SYK; STY; TEK; ERK; TIE; TKF; NTRK3; MLK3; PRKM4; PRKM1; PTK7; EEK; MNBH; BMX; ETK1; MST1R; 135 KD BTK-ASSOCIATED PROTEIN; LCK; FGFR2; TYK3; FER; TXK; TEC; TYK2; EPLG1; EMT; EPHT1; ZRK; PRKMK1; EPHT3; GAS6; KDR; AXL; FGFR1; ERBB2; FLT3; NEP; NTRKR3; EPLG5; NTRK2; RYK; BLK; EPHT2; EPLG2; EPLG7; JAKI; FLT1; PRKAR1A; WEE1; ETK2; MuSK; INSR; JAK3; FMS-related tyrosine kinase-3 LIGAND; PRKCB1; HER3; JAK2; LIMK1; DUSP1; DMD; HCK; YWHAH; RET; YWHAZ; YWHAB; HTK; MAP Kinase Kinase 6; PIK3CA; CDKN3; Diacylglycerol Kinase; PTPN13; ABL1; DAGK1; Focal Adhesion Kinase 2; EDDR1; ALK; PIK3CG; PIK3R1; EHK1; KIT; FGFR3; VEGFC; MST1; FHC; EGFR; S100A10; NF1; TRK; CML; GRB7; S100A4; RASA2; MET; STAT3; smg GDS-Associated Protein; Ubiquitin-Binding Protein P62; LCP2; EPS15; GRB10; GDNFRA; SHC1; CF; TPM3; CDC2; LGMD2C; Ash Protein; TSD; AGRN; S100A6; HPRT1; Cytovillin; GLG1; GRB14; FES; P32 Splicing Factor SF2 Associated Protein; Cartilage-Derived Morphogenetic Protein 1; PAX5; IRS1; SOS2; PIGA; RHO; TGFBR2; CSF1R; PDNP1; NPM1; ADDI; HMMR; ESR; SLA; PGF; ETV6; M6P2; FGR; FGF8; SNX1; TCF1; HGF; IL6R; YESI; ENG; HCLS1; GTF2H1; PDGFB; PDCD1; TGFBR1; EPS8; VEGF; CAR; ANGPT2; Hypogammaglobulinemia And Isolated Growth Hormone Deficiency, X-LINKED; Glial Cell Line-Derived Neurotrophic Factor Receptor-BetA; and H4 gene and mutants and/or variants thereof.

Preferably, the physiological response database comprises information relating not only to the expression of GPCR pathway biomolecules, but also includes information relating to the biological impact of this expression. For example, the database 5 preferably includes information relating the expression of a plurality of GPCR pathway biomolecules to physiological responses to disease, pathological conditions, drugs, agents, therapies, environmental conditions, and the like. The database can also include information relating the expression of GPCR pathway biomolecules to physiological parameters such as blood pressure, heart rate, pH, body temperature, level of metabolites, and the like. In some aspects, information relating to biological impact includes the association of the expression of GPCR pathway biomolecules with parameters considered as being important to quality of life, e.g., levels of pain, ability to move, sleep, eat, and the like.

Preferably, a control subdatabase also is provided comprising information relating to the average physiological responses of healthy patients in specific demographic groups. This database can further include information relating to the expression of housekeeping genes in different tissues and different stages of development.

Still more preferably, the database also links information relating to the expression of GPCR pathway molecules to information about patient characteristics. For example, in one aspect, the database includes information relating to the sources of tissues on a plurality of microarrays which have been evaluated to determine the expression of a plurality of GPCR pathway biomolecules. This information can include, but is not limited to, information regarding the age, sex, weight, height, ethnic background, occupation, environment, family medical background and medical history of the sources of the tissue samples on the microarray. Medical history information can include information pertaining to prior and current diseases or conditions, diagnostic and prognostic test results, drug exposure, or exposure to other therapeutic agents, responses to drug exposure or exposure to other therapeutic agents, history of alcoholism, drug or tobacco use, cause of death, if appropriate, and the like.

In one aspect, the physiological response database 5 includes information relating to the effect of drugs on a plurality of GPCR pathway biomolecules and/or information relating to the localization of one or more drugs in tissues on a whole body microarray from one or more patients. Subdatabases including this information can be organized according to particular classes of drugs and particular concurrent and underlying illnesses which a patient has experienced or is experiencing or according to other common patient characteristics. In some aspects, the drugs correlated to physiological responses include anti-cancer agents such as those described in Weinstein et al., Science 258: 447 (1992) and van Osdol et al, J. Natl. Cancer Inst. 86: 1853 (1994) and/or compounds included in an external database such as the Anti-Cancer Agent Mechanism Database at http://dtp.nci.nih.gov/docs/cancer/searches/standard_agent.html. Still other subdatabases can be provided in which the expression of GPCR pathway biomolecules is correlated with exposure of a patient to one or more toxic agents and/or environmental conditions.

In a further aspect, the physiological response database comprises a database of information relating to treatment options, including, but not limited to drugs available to patients who exhibit particular physiological responses. Treatment databases can further include expert rules for correlating particular treatment options to particular physiological responses. Treatment databases are known in the art and are described in U.S. Pat. No. 6,188,988, for example, the entirety of which is incorporated by reference herein.

Information Management System For Identifying GPCR Pathway Biomolecules and For Modeling GPCR Pathways

The database 5 according to the invention is coupled to an Information Management System (IMS) 7. In one aspect, the IMS 7 includes functions for searching and determining relationships between data structures in the database 5. In another aspect, the IMS 7 displays information obtained in this process on an interface 6 of the user device 3. In one aspect, the IMS 7 is stored within one or more servers 4, and is accessible remotely by the user of the device 3 through the network 2. In another aspect of the invention, the IMS 7 is accessible through a readable medium, which the user accesses through their particular device 3, such as a CD-ROM.

IMS 7's encompassed within the scope of the present invention include the Spotfire™ program, which is described in U.S. Pat. No. 6,014,661, the entirety of which is incorporated by reference herein. This database management software provides links to genomics data sources and those of key content and instrumentation providers, as well as providing computer program products for gene expression analysis. The software also provides the ability to communicate results and records electronically. Other programs can also be used, and are encompassed within the scope of the invention, and include, but are not limited to Microsoft Access, ORACLE and ILLUSTRA. Java-based applications also can be used to facilitate management of large datasets.

In one aspect, the IMS 7 comprises a stored procedure or programming logic stored and maintained by the IMS 7. Stored procedures can be user-defined, for example, to implement particular search queries or organizing parameters. Examples of stored procedures and methods of implementing these are described in U.S. Pat. No. 6,112,199, the entirety of which is incorporated herein by reference.

In one aspect of the invention, the IMS 7 includes a search function which provides a Natural Language Query (NLQ) function. In this aspect, the NLQ accepts a search sentence or phrase in common everyday from a user (e.g., natural language inputted into an interface of a device 3) and parses the input sentence or phrase in an attempt to extract meaning from it. For example, a natural language search phrase used with the specimen-linked database 5, could be “provide medical history of patient at sublocation 1,1 of microarray 4591.” This sentence would processed by the search function of the IMS 7 to determine the information required by the user which is then retrieved from the specimen-linked database 5. In another aspect of the invention, the search function of the IMS 7 recognizes Boolean operators and truncation symbols approximating values that the user is searching for.

In one aspect, the search function of the IMS 7 generates search data from terms inputted into a field displayed on an interface 6 of a device 3 in the system 1 in a form recognized by at least one search engine (e.g., identifying search terms which are stored in fields in the database 5 or in the summary subdatabase) and transfers the search data to at least one search engine to initiate a search. However, in another aspect, the search query is communicated through the selection of options displayed on the interface 6. For example, in one aspect, search results are displayed on the interface 6, which may be in the form of a list of information sources retrieved by the at least one search engine. In another aspect, the list comprises links which link the user to information provided by the information source. In a further aspect, the search function of the IMS 7 removes redundancies from the list and/or ranks the information sources according to the degree of match between the information source and the search terms extracted, and the interface 6 displays the information sources in order of their rankings. Search systems which can be used are described in U.S. Pat. No. 6,078,914, for example, the entirety of which is incorporated by reference herein.

In another aspect, the search function of the IMS 7 searches a summary subdatabase of the database 5 to identify particular subdatabase(s) most relevant to the search terms which have been inputted by the user. In this aspect, the search function of the IMS 7 restricts its search to subdatabases so-identified. In a further aspect, the subdatabases searched by the IMS 7 can be defined by the user.

In one aspect, relationships are defined by codes, such as SNOMEDO codes, which can be inputted into the system by a user (e.g., on an interface of a user device). SNOMED® and SNOMED codes are described further in Altman et al., Proceedings of American Medical Informatics Association Eighteenth Annual Symposium on Computer Applications in Medical Care. November 5-9, Washington D.C. pg. 179-183; Bale, Pathology.; 23(3): 263-267, 1991; Ball, et al., Computing pp. 40-46, 1999; Barrows, et al., Proceedings of American Medical Informatics Association Eighteenth Annual Symposium on Computer Applications in Medical Care, November 5-9, Washington D.C. pg. 211; Beckett, Pathologist, Vol. XXXI, No. 7, July 1977; Bell, Journal of the American Medical Informatics Association, 1(3): 207-217 (1994); Benoit et al., Proceedings of the Annual Symposium of Computers Applications in Medical Care. 1992; pp. 787-788; Berman, et al., A SNOMED Analysis of Three Years' Accessioned Cases (40,124) of Surgical Pathology Department: Implications for Pathology-based Demographic Studies. Proceedings of American Medical Informatics Association Eighteenth Annual Symposium on Computer Applications in Medical Care. Nov. 5-9, 1994, Washington D.C. pg. 188-192; Berman, et al., Modern Pathology. 9(9): 944-950 (1996); Bidgood,. Meth. Inf. Med. 37: 404-414 (1998); Brigl et al., International Journal of Bio-Medical Computing. 38: 101-108 (1995); Brigl et al., Int J Biomed Comput. 37(3): 237-247 (1994); Campbell et al., Methods Inf. Med. 37 (4-5): 426-39 (1998); and Campbell et al., Proceedings of American Medical Informatics Association Eighteenth Annual Symposium on Computer Applications in Medical Care. Nov. 5-9, 1994, Washington, D.C. pg. 201-205, for example, the entireties of which are incorporated by reference herein.

In a further aspect of the invention, the IMS-7 includes a mapping function for mapping terms to particular tables within the database 5. Alternatively, or in addition to SNOMED®, other classification and mapping codes can be used (e.g., CPT, OPCS-4, ICD-9, and ICD-10). In one aspect, the IMS-7 comprises a program enabling it to read inputted codes and to access and display appropriate information from a relationship table. For example, in one aspect, unique SNOMED® codes are assigned to tissues from specific anatomic sites, while in another aspect, codes are assigned to tissues having specific pathologies (e.g., specific types of cancer) and/or having selected pathologies (e.g., diagnostic codes are assigned to tissue samples/specimens which are the targets of specific types of cancer). In a further aspect (not shown), tissue samples/specimens are cross-referenced using SNOMED® codes for both anatomic sites and diagnosis. Exposure of individual tissue samples to particular drugs can also be indicated by codes such as by using American Hospital Formulary Service List (AHFS) Numbers or “V-Codes” to classify other types of circumstances or events to which the source of a tissue sample has been exposed such as vaccinations, potential health hazards related to personal and family history, and exposure to toxic chemicals, and the like (see, e.g., as described in U.S. Pat. No. 6,113,540, the entirety of which is incorporated by reference herein).

In a further aspect, specimens/tissues are obtained from individuals having a neuropsychiatric disorder, and specimens/tissues on a microarray are cross-referenced in the database (i.e., linked to the database) according to the individuals' classification using DSM-IV-TR criteria. In another aspect, specimens/tissues are linked to the database using ICD-9-CM criteria. In still another aspect, the specimens/tissues are cross-referenced using a number of criteria, such as tissue type, date of birth of the source individual, medical history of the source individual, ICD-9 criteria, DSM-IV TR criteria, Medications, and method of preparation. In a further aspect, the ICD-9 and/or DSM-IV-TR criteria are indicated using codes. ICD-9 and DSM-IV TR codes are described at http://www.nzhis.govt.nz/projects/dsmiv-code-table.html, for example.

In addition to comprising a search function, the IMS 7 comprises a relationship determining function. In one aspect, in response to a query and/or the user inputting information regarding a tissue into the tissue information system 1, the IMS 7 searches the database 5 and classifies tissue information within the database 5 by type or attribute (e.g., patient sex, age, disease, exposure to drug, tissue type, cancer grade, cause of death, and the like, and/or by codes, such as by SNOMED® codes, ICD-9 codes, and/or DSM-IV-TR codes). In one aspect, when all attributes have been defined and classified as characteristic of defined relationship(s), the IMS 7 assigns a relationship identification number to each attribute, or set of attributes, and signals representing these attribute(s) are stored in the database 5 (e.g., as part of the data dictionary subdatabase) where they are indexed by the relationship ID# and provided with a descriptor. For example, in one aspect, the expression of a plurality of biological characteristics which have been classified as correlating to a disease state X (e.g., cancer) is assigned an ID# and a descriptor such as “diagnostic traits of disease X.”

In one aspect, the relationship determining function of the IMS 7 employs a statistical program to identify groups of attributes as representing a particular relationship. In one aspect, the statistical program is a non-hierarchical clustering program. In another aspect, the clustering program employs k-means clustering.

Clustering programs can also be used to identify structural relationships between newly identified pathway molecules to identify conserved domains and similar structures. The identification of conservation can be used to establish initial predictions regarding interactions between candidate pathway molecules and other pathway molecules based on the existence of such interactions in other organism. In one aspect, the IMS-7 is used in conjunction with one or more genomic and/or proteonomic database and search plateforms, including, but not limited to GeneData Phylosopherm, GeneSpring™ (available from Silicon Genetics), MetaMine™, and the like. Such platforms are intended to complement the IMS-7 system's ability to access and perform operations on disparate data.

Pipelining can be used to streamline various operations performed by the IMS-7 allowing disparate data sources to be analyzed sequentially and allowing data to be screened using characteristics not necessarily stored in the database.

The IMS 7 analyzes the relationships between data in the database 5 and/or new data being inputted, using any method standardly used in the art, including, but not limited to, regression, decision trees, neural networks, and fuzzy logic, and combinations thereof. In response to the results of this analysis, upon a query by a user, the system 1 displays at least one relationship or identifies that no discernable relationship can be found on the interface 6 of the user device 3. In one aspect, the system 1 displays descriptors relating to plurality of relationships identified by the IMS 7 on the interface 6 as well as information relating to the statistical probability that a given relationship exists.

In one aspect, the user selects among a plurality of relationships identified by the IMS 7 by interfacing with the interface 6 to determine those of interest (e.g., a relationship which is a disease correlation might be of interest, while a relationship regarding hair color might not be). In another aspect of the invention, rather than scanning an entire database 5, the IMS 7 samples the database 5 randomly until at least one statistically satisfactory relationship is identified, with the user setting parameters for what is “statistically satisfactory.” In a further aspect of the invention, the user identifies particular subdatabases for the IMS 7 to search. In still another aspect, the IMS 7 itself identifies particular subdatabases based on query terms the user of the system 1 has provided.

In one aspect of the invention, the relationship of interest is used to provide a diagnosis or prognosis of a disease (e.g., the relationship identified is a high correlation with a disease state or with the progression of a disease). In another aspect of the invention, the relationship of interest is used to identify the biological role of an uncharacterized gene, or to identify particular demographic factors (e.g., such as socioeconomic factors) associated with a disease state or other physiological response to a condition.

In one aspect of the invention, the IMS-7 system is used to identify populations of patients who share selected clinical characteristics by identifying sources of tissue samples who have these clinical characteristics. Clinical characteristics may be embodied in data which has already been entered into the database 5 or may be embodied in new data, which is being inputted into the system for validation. In one aspect, populations of patients are identified who share a particular clinical history or outcome, a specific type of physiological response to a drug, either adverse or beneficial.

In another aspect, the IMS-7 identifies relationships between sets of genes expressed or not expressed in tissues on one or more microarrays and clinical information relating to the patients from whom the tissues were obtained. For example, in one aspect, the IMS-7 identifies relationships between a pathological condition (e.g., such as stroke) and genes expressed or not expressed during in tissues from patients who have experienced or are experiencing the condition. For example, in one aspect, the relationship determining function of the IMS-7 (for example, an application program which performs k-means clustering) is used to designate potential GPCR pathway genes, i.e., genes which are expressed during a disease and whose expression is related to the expression of other genes in a particular GPCR pathway.

Thus, in a very simple aspect, where a stroke victim A expresses genes 1, 2, 3, 4, a stroke victim B expresses genes 1, 2, 4,7, 8, a stroke victim C expresses genes 1, 2, 4, 8, 9, 10, and normal patients D, E, and F express genes 2, 3, 8, the IMS 7 would identify genes 1, 4, 7, 9, and 10 as potentially involved in a pathway of genes affected during stroke, and in certain aspects, would rank genes 1 and 4 as being highly likely to be pathway genes. In a further aspect, the IMS 7, in response to a user query would identify other patient parameters associated with the expression of genes 7, 9, and 10 and would perform clustering analyses to determine whether any relationships identified were statistically unlikely to arise by chance. For example, the IMS 7 might identify that populations expressing genes 7, 9, and 10, in addition to stroke, suffer from cardiovascular disease.

In one aspect of the invention, the IMS 7 includes an expert system. For example, the IMS 7 can comprise an object-oriented deployment system (e.g., such as the G2 Version 3.0 Real Time Expert System, available from Gensym, Corp.). Static Expert systems can also be used. Expert systems can be used to establish rules and procedures to identify and validate molecular pathways and to correlate changes in the expression of GPCR pathway biomolecules with any of the physiological responses described above. In one aspect, the expert system includes an inference function that operates on information within the specimen-linked database 5 and its associated subdatabases to identify biomolecules which are likely to belong to a GPCR pathway. The inference function allows the system 1 to rank pathways identified according to their probability of occurrence given the information which has been inputted into the database 5. In other aspects, the system 1 can be directed by a user to simulate GPCR pathways and to compare these pathways with molecular profiling data within the database 5. Preferably, the IMS 7 ranks simulated pathways according to their likelihood of occurrence based on data obtained from a plurality of tissue microarrays. The expert system of the IMS 7 can further include a transaction manager whose function is to direct input and output requests between one or more servers 4 of the system 1 and the interfaces of one or more user devices 3 of the system, in order to respond to user requests.

Expert systems are known in the art and include such systems as MYCIN, EMYCIN, NEOMYCIN, and HERACLES (see, e.g., Clancy, “From Guidon to Neomycin and Heracles in Twenty Short Lessons: ORN Final Report 1979-1985,” The AI Magazine 8/86, pp. 40-60; Thompson et al., “A Qualitative Modeling Shell for Process Diagnosis,” 1986 IEEE Software, pp. 6-15; Bylander, “CRSL: A Language for Classificatory Problem Solving and Uncertainty Handling,” The AI Magazine 8/86, pp. 66-77; Hofmann et al., “Building Expert Systems for Repair Domains,” Expert Systems, 1/86, vol. 3, No. 1, pp. 4-11; and Yung-Choa Pan et al., “Pies: A Engineer's Do-It-Yourself Knowledge System for Interpretation of Parametric Test Data,” AI Magazine, Fall, 1986, pp. 62-69). Other expert systems are described in, for example, U.S. Pat. No. 6,154,750, U.S. Pat. No. 6,188,988, U.S. Pat. No. 6,149,585, U.S. Pat. No. 6,055,507, U.S. Pat. No. 5,991,730, and U.S. Pat. No. 5,777,888, and U.S. Pat. No. 4,866,635. The entireties of these references are incorporated by reference herein.

Relationships identified by the IMS 7 can be displayed to the user in a variety of formats such as graphs, histograms, dendograms, charts, tables and the like. In a preferred aspect, in response to a request by a user, the system 1 displays on the interface of a user device 3 a representation of a molecular pathway which includes a plurality of GPCR pathway biomolecules graphically arranged according to their effect on the expression of other biomolecules within the same GPCR pathway (e.g., connected by arrows and the like). When a user selects a particular GPCR pathway biomolecule on the “pathway interface” (e.g., by moving a cursor to a representation of the biomolecule, such as the biomolecule's name), the user is linked to an interface which provides information relating to the biomolecule. The interface can alternatively, or additionally, provide information category links which provide the user with access to portions of the database 5 which comprise information related to a particular information category.

Information about a biomolecule can include a three-dimensional molecular structure information, sequence information and/or links to external genomic and/or protein databases, where appropriate (e.g., such as GenBank or SWISS-Prot), information relating to one or more of: mutations, allelic variants, ligands, substrates, products, cofactors, agonists, and antagonists, reference links to external databases including references about the biomolecule (e.g., PubMed), and information about available clones (e.g., cDNA molecules expressing a pathway protein), if applicable, and the like.

In a preferred aspect, the user can access an “expression profile interface” on which is displayed a representation of the levels and/or forms of expression of the selected GPCR pathway biomolecule in a plurality of tissues. Preferably, this interface is also associated with one or more information category links identifying physiological response categories such as responses to diseases, pathological conditions, drugs or other agents, environmental conditions and the like. Selecting one of these information categories will link the user to an interface on which is displayed an expression profile of the biomolecule during a particular physiological response. In certain aspects, the expression profiles of GPCR pathway molecules in a plurality of tissues during a plurality of different physiological responses is displayed on a single interface for comparison. In one aspect, in response to a user query, the system performs an electronic subtraction analysis and displays differences in expression profiles on a single interface. Electronic subtraction methods are known in the art (see, for example, U.S. Pat. No. 6,114,114, the entirety of which is incorporated by reference herein). A “pathway home” button can be provided on any or all of these interfaces to direct a user back to the interface displaying the pathway.

In one aspect, selecting a GPCR pathway biomolecule on a pathway interface provided by the system 1 displays a pull down menu which provides the user with the simulation options, such as “delete,” “underexpress” and/or “overexpress.” Selecting one of these options directs the IMS 7 to simulate the effects of deleting, underexpressing and/or overexpressing the biomolecule identified on the expression of other biomolecules in the GPCR pathway. In some aspects, selecting “underexpress” or “overexpress” causes a pull down menu of values to be displayed (e.g., 2× or −2×; selecting 2× would show the effects of doubling the biomolecule, while selecting −2× would show the effects of halving the biomolecule). In some aspects, the system 1 is used to model the effect of one or more feedback loops on the pathway.

In some aspects, selecting a representation of a GPCR receptor in a pathway interface links the user to an interface which displays information categories links relating to “antagonists” and “agonists” of the receptor molecule. These links provide a user with access to portions of the specimen-linked database which include information relating to molecules which have been demonstrated to alter the interaction of the receptor with its ligand. These molecules can include drugs with known dissociation constants and characterized circulating half lives. However, in other aspects, the user can direct the IMS 7 to simulate the molecular structure of antagonist or agonist molecule and model the effect of binding such a molecule to the receptor on the expression of other pathway molecules in the pathway to which the receptor belongs. In silico modeling of receptor ligand interactions is known in the art and is described in, for example, Lengauer et al., Curr. Opin. Struct. Biol. 5: 402-406 (1996); Strynadka et al., Nature Struct. Bio. 3: 233-239 (1996); Chen et al., Biochemistry 36: 11402-11407 (1997); and Kuntz, et al., J. Mol. Biol. 161: 269-288 (1982); the entireties of which are incorporated by reference herein. In one aspect, the Viseur program (see, e.g., Campagne et al., J. Comput. Aided Mol. Des. 13(6): 625-643 (1999), the entirety of which is incorporated by reference herein) is used to model a GPCR and to link the specimen-linked database with a mutagenesis data server (e.g., such as the GPCRDB server) to model interactions between wild type and variant GPCRs and peptide mimetics, agonists, and antagonists. Automated GPCR modeling systems are also available through the Internet at http://expasy.hcuge.ch/swissmod/SWISS-MODEL.+++html.

In some aspects, the IMS 7 is used to identify the effects of agents (e.g., mimetics, antagonists, agonists or potentially toxic agents) on a plurality of GPCR pathway molecules by comparing the physiological responses of cells in culture exposed to one or more agents with the biological characteristics of samples of these cells arrayed on tissue microarrays. Thus, in some aspects, the IC₅₀ value, or the concentration of an agent that causes 50% growth inhibition, the GI₅₀ value (which measures the growth inhibitory effect of an agent) the TGI (which provides a measure of an agent's cytostatic effect), and/or the LC₅₀ (which provides a measure of the agent's cytotoxic effect) is measured in vitro and correlated with the expression of one or more GPCR pathway biomolecules in samples on microarrays. In the case of agonists or antagonists, the effects of these agents on dissociation constants and other kinetic parameters of GPCRs can also be measured.

In some aspects, in response to a user query, the system 1 displays a “mean graph” interface or an interface which provides a display of the pattern created by plotting positive and negative values generated from a set of GI₅₀, TGI, or LC₅₀ values. For example, positive and negative values can be shown plotted along a vertical line that represents the mean response of all cells exposed to an agent. Positive values provide a measure of which cellular sensitivities are significant, while negative values indicate results that are not significant. Mean graphs are described in, for example, Paull et al., J. Natl. Cancer Inst. 81: 1088-1092 (1989);. Paull et al., Proc. Am. Assoc. Cancer Res. 29: 488 (1988), the entireties of which are incorporated by reference herein.

In some aspects, the IMS 7 implements a COMPARE algorithm to provide an ordered list of agents ranked according to their effects on the physiological responses of cells and/or tissues and on the expression of GPCR pathway biomolecules in these cells and/or tissues. COMPARE algorithms are described in Paul et al., supra, and in Hodes et al., J. Biopharm. Stat. 2: 31-48 (1992), the entireties of which are incorporated by reference herein. Data obtained from this analysis can be added to the specimen-linked database 5 and made available to other users of the system 1. The IMS 7 also can include statistical programs to facilitate comparisons such as PROC CORR. Other algorithms, such as the DISCOVER algorithm also can be used.

In a preferred aspect, in response to a user query, the system 1 will display an interface which includes a representation of the expression profiles of GPCR pathway biomolecules in tissues exposed to an agent characterized as described above. In still more preferred aspects, the system 1 will perform an electronic subtraction to show only changes in expression profiles in treated tissues compared to untreated tissues. In still other aspects, changes in expression values are expressed as ratios of differences (e.g., level of biomolecule A in treated tissue 1/level of biomolecule A in untreated tissue 1) or as percent changes of expression.

The above assays can be performed in parallel with assays using animals who have also been exposed to the same agents to compare the physiological responses of these animals with the expression of GPCR pathway biomolecules in whole body tissue microarrays obtained from these animals. Physiological responses measured can include the overall health of the animal, organ function, levels of metabolites and other molecules in the blood, behavioral changes, and the like. In some aspects, the localization of the agents in tissues on the microarrays is determined, for example, by using labeled aptamer probes or other molecular probes which recognize these agents.

Similarly, the physiological responses of patients to agents can also be correlated with the expression of a plurality of GPCR pathway biomolecules by using tissue microarrays. In some aspects, patient samples are derived from autopsies and the expression of GPCR pathway biomolecules in whole body tissue microarrays is correlated with detailed information relating to the patient's medical history (e.g., including drug exposure), family medical history, and other characteristics which have been inputted into the specimen-linked database 5.

In one aspect, the user is able to view, print, permanently store, read, and/or further manipulate data displayed on the display 6 of his or her device 3. In this aspect, the user is able to use the system 1 to investigate and define the relationships most relevant to tissues or diseases of interest. In one aspect, the user is also able to link to any database publicly accessible through the network 2, and to integrate information from such a database with the system 1's database 5 through the IMS 7. Thus, in one aspect, information can be shared with other users and information from other users can be continuously added to the database 5.

One aspect of the invention recognizes potential difficulties in enabling unrestricted access to the database 5, and encompasses providing restricted access to the database 5, and/or restricted ability to change the contents of the database 5 or records in the database 5 using the IMS 7 and/or a security application. Methods of providing restricted access to electronic data are known in the art, and are described, for example, in U.S. Pat. No. 5,910,987, the entirety of which is incorporated by reference herein.

Molecular Probes

Antibodies For Detection of Biological Characteristics

Antibodies specific for a large number of known antigens are commercially available. Links to multiple antibody suppliers can also be found at http://www.antibodyresource.com/misc.html. When antibodies are not commercially available, one of skill in the art can readily raise their own antibodies using standard techniques.

In order to produce antibodies, various host animals are immunized by injection with the growth-related polypeptide or an antigenic fragment thereof. Useful animals include, but are not limited to rabbits, mice, rats, goats, and sheep. Adjuvants may be used to increase the immunological response to the antigen. Examples include, but are not limited to, Freund's adjuvant (complete and incomplete), mineral gels such as aluminum hydroxide, surface active substances such as lysolecithin, pluronic polyols, polyanions, peptides, oil emulsions, keyhole limpet hemocyanin, dinitrophenol, and adjuvants useful in humans, such as BCG (bacille Calmette-Guerin) and Corynebacterium parvum. These approaches will generate polyclonal antibodies.

Monoclonal antibodies specific for a polypeptide may be prepared using any technique that provides for the production of antibody molecules by continuous cell lines in culture. These include, but are not limited to, the hybridoma technique originally described by Kohler and Milstein, Nature 256: 495-497 (1975); the human B-cell hybridoma technique (Kosbor et al., Immunology Today 4: 72 (1983); Cote et al., Proc. Natl. Acad. Sci. U.S.A. 80: 2026-2030 (1983)) and the EBV-hybridoma technique (Cole et al., Monoclonal Antibodies and Cancer Therapy, Alan R. Liss, Inc., pp. 77-96. (1985)). In addition, techniques developed for the production of “chimeric antibodies” (Morrison et al., Proc. Natl. Acad. Sci. U.S.A. 81: 6851-6855 (1984)); Neuberger et al., Nature 312:604-608 (1984); Takeda et al., Nature 314:452-454 (1985)) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce growth-related polypeptide-specific single chain antibodies. The entireties of these references are incorporated by reference herein.

Antibody fragments which contain specific binding sites of a growth-related polypeptide may be generated by known techniques. For example, such fragments include, but are not limited to, F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., Science 246:1275-1281 (1989)) to allow rapid and easy identification of monoclonal Fab fragments with the desired specificity to a growth-related polypeptide. An advantage of cloned Fab fragment genes is that it is a straightforward process to generate fusion proteins with, for example, green fluorescent protein for labeling.

Antibodies, or fragments of antibodies may be used to quantitatively or qualitatively detect the presence of growth-related polypeptides or conserved variants or peptide fragments thereof. For example, immunofluorescence techniques employing a fluorescently labeled antibody coupled with light microscopic, or fluorimetric detection can be used.

Antibodies or antigen binding portions thereof may be employed histologically, as in immunohisto chemistry, immunofluorescence, immunoelectron microscopy, or an histological assays, for in situ detection of polypeptides or other antigen-containing biomolecules.

### Allele-Specific Antibodies and Modification-Specific Antibodies

In preferred aspects, antibodies are used which are specific for specific allelic variants of a protein or which can distinguish the modified from the unmodified form of a protein (e.g., such as a phosphorylated vs. an unphosphorylated form or a glycosylated vs. an unglycosylated form of a polypeptide). For example, peptides comprising protein allelic variations can be used as antigens to screen for antibodies specific for these variants. Similarly modified peptides or proteins can be used as immunogens to select antibodies which bind only to the modified form of the protein and not to the unmodified form. Methods of making allele-specific antibodies and modification-specific antibodies are known in the art and described in U.S. Pat. No. 6,054,273; U.S. Pat. No. 6,054,273; U.S. Pat. No. 6,037,135; U.S. Pat. No. 6,022,683; U.S. Pat. No. 5,702,890; U.S. Pat. No. 5,702,890, and in Sutton et al., J. Immunogenet 14(1): 43-57 (1987), the entireties of which are incorporated by reference herein.

Immunohistochemistry (IHC)

In situ detection of an antigen can be accomplished by contacting a test tissue and microarray on a profile array substrate with a labeled antibody that specifically binds the antigen. The antibody or antigen binding portion thereof is preferably applied by overlaying the labeled antibody or antigen binding portion onto the test tissue and microarray. Through the use of such a procedure, it is possible to determine not only the presence of the antigen but also its amount and its localization in a test tissue and in the plurality of sublocations within the microarray.

In one aspect, antibodies are detectably labeled by linkage to an enzyme for use in an enzyme immunoassay (EIA) (Voller, Diagnostic Horizons 2: 1-7 (1978), Microbiological Associates Quarterly Publication, Walkersville, Md.); Voller et al., J. Clin. Pathol. 31: 507-520 (1978); Butler, Meth. Enzymol. 73: 482-523 (1981); Maggio, E. (ed.), 1980, In Enzyme Immunoassay, CRC Press, Boca Raton, Fla.; Ishikawa et al., (eds.), 1981, In Enzyme Immunoassay, Kgaku Shoin, Tokyo). The enzyme which is linked to the antibody will react with an appropriate substrate, preferably a chromogenic substrate, in such a manner as to produce a chemical moiety which is detectable, for example, by spectrophotometric, fluorimetric or visual means. Examples of enzymes useful in the methods of the invention include, but are not limited to peroxidase, alkaline phosphatase, and RTU AEC.

Detection of bound antibodies can alternatively be performed by radiolabeling antibodies and detecting the radiolabel. Following binding of the antibodies and washing, the samples may be processed for autoradiography to permit the detection of label on particular cells in the samples.

In one aspect, antibodies are labeled with a fluorescent compound. When the fluorescently labeled antibody is exposed to light of the proper wavelength, its presence can be detected due to fluorescence. Many fluorescent labels are known in the art and may be used in the methods of the invention. Preferred fluorescent labels include fluorescein, amino coumarin acetic acid, tetramethylrhodamine isothiocyanate (TRITC), Texas Red, Cy3.0 and Cy5.0. Green fluorescent protein (GFP) is also useful for fluorescent labeling, and can be used to label non-antibody protein probes as well as antibodies or antigen binding fragments thereof by expression as fusion proteins. GFP-encoding vectors designed for the creation of fusion proteins are commercially available.

The primary antibody (the one specific for the antigen of interest) may alternatively be unlabeled, with detection based upon subsequent reaction of bound primary antibody with a detectably labeled secondary antibody specific for the primary antibody. Another alternative to labeling of the primary or secondary antibody is to label the antibody with one member of a specific binding pair. Following binding of the antibody-binding pair member complex to the sample, the other member of the specific binding pair, having a fluorescent or other label, is added. The interaction of the two partners of the specific binding pair results in binding the detectable label to the site of primary antibody binding, thereby allowing detection. Specific binding pairs useful in the methods of the invention include, for example, biotin:avidin. A related labeling and detection scheme is to label the primary antibody with another antigen, such as digoxigenin. Following binding of the antigen-labeled antibody to the sample, detectably labeled secondary antibody specific for the labeling antigen, for example, anti-digoxigenin antibody, is added which binds to the antigen-labeled antibody, permitting detection.

The staining of tissues for antibody detection is well known in the art, and can be performed with molecular probes including, but not limited to, AP-Labeled Affinity Purified Antibodies, FITC-Labeled Secondary Antibodies, Biotin-HRP Conjugate, Avidin-HRP Conjugate, Avidin-Colloidal Gold, Super-Low-Noise Avidin, Colloidal Gold, ABC Immu Detect, Lab Immunodetect, DAB Stain, ACE Stain, NI-DAB Stain, polyclonal secondary antibodies, biotinylated affinity purified antibodies, HRP-labeled affinity purified antibodies, and/or conjugated antibodies.

In one aspect, immunohistochemistry is performed using an automated system such as the Ventana ES System and Ventana gen^{II}™ System (Ventana Medical Systems, Inc., Tucson, Ariz.). Methods of using this system are described in U.S. Pat. No. 5,225,325, U.S. Pat. No. 5,232,664, U.S. Pat. No. 5,322,771, U.S. Pat. No. 5,418,138, and U.S. Pat. No. 5,432,056, the entireties of which are incorporated by reference herein.

Nucleic Acid Probes

Nucleic acid probes can also be used where the sequence of a gene encoding a biomolecule is known. Means for detecting specific DNA sequences within genes are well known to those of skill in the art. In one aspect, oligonucleotide probes chosen to be complementary to a selected subsequence within the gene can be used. Nucleic acid probes can be fragments of larger nucleic acid molecules (e.g., such as obtained by restriction enzyme digestion or by PCR or another amplification technique) or can be synthetic molecules. Modified nucleic acids (e.g., comprising one or more altered bases, sugars, and/or internucleotide linkages) and analogs (e.g., such as PNA molecules) are also encompassed within the scope of the invention.

Methods of labeling nucleic acids are well known to those of skill in the art. Preferred labels are those that are suitable for use in in situ hybridization (ISH) or fluorescent in situ hybridization (FISH). In one aspect, nucleic acid probes are detectably labeled prior to hybridization with a tissue sample. Alternatively, a detectable label which binds to the hybridization product can be used. Labels for nucleic acid probes include any composition detectable by spectroscopic, photochemical, biochemical, immunochemical, or chemical means and include, but are not limited to, radioactive labels (e.g. ³²P, ¹²⁵I, ¹⁴C, ³H, and ³⁵S), fluorescent dyes (e.g. fluorescein, rhodamine, Texas Red, etc.), electron-dense reagents (e.g. gold), enzymes (as commonly used in an ELISA), colorimetric labels (e.g. colloidal gold), magnetic labels (e.g. Dynabeads TM), and the like. Examples of labels which are not directly detected but are detected through the use of directly detectable label include biotin and dioxigenin as well as haptens and proteins for which labeled antisera or monoclonal antibodies are available.

A direct labeled probe, as used herein, is a probe to which a detectable label is attached. Because the direct label is already attached to the probe, no subsequent steps are required to associate the probe with the detectable label. In contrast, an indirect labeled probe is one which bears a moiety to which a detectable label is subsequently bound, typically after the probe is hybridized with the target nucleic acid.

Labels can be coupled to nucleic acid probes in a variety of means known to those of skill in the art. In some aspects the nucleic acid probes are labeled using nick translation or random primer extension (Rigby et al. J. Mol. Biol., 113: 237 (1977) or Sambrook et al., Molecular Cloning: A Laboratory Manual, Cold Spring Harbor Laboratory, Cold Spring Harbor, N.Y., (1989), the entireties of which are incorporated by reference herein).

Alternatively, sequences or subsequences of tissues within a microarray may be amplified by a variety of DNA amplification techniques (e.g., polymerase chain reaction, ligase chain reaction, transcription amplification, etc.) prior to detection using a probe. Amplification of nucleic acid sequences increases sensitivity by providing more copies of possible target subsequences. In addition, by using labeled primers in the amplification process, the sequences are labeled as they are amplified.

Aptamer Probes

Aptamer probes are also encompassed within the scope of the invention, e.g., to label molecules which are not readily bound by nucleic acids using Watson-Crick binding or by antibodies. Methods of generating aptamers are known in the art and described in U.S. Pat. No. 6,180,406 and U.S. Pat. No. 6,051,388, for example, the entireties of which are incorporated by reference herein. Aptamers can generally be labeled as described above with reference to nucleic acid probes.

### In Situ Hybridization (ISH) and Fluorescent In Situ Hybridization (FISH)

In situ hybridization (ISH) and Fluorescent In Situ Hybridization (FISH) are techniques that can avail themselves to paraffin-embedded sectioned tissue. Both techniques are genomic based rather than proteomic based, as in IHC, and involve RNA and DNA probes that will hybridize, or specifically bind to their complement base sequence. In some aspects, labels are attached to genomic probes that allow hybridization of the probes to be visualized under a microscope. ISH probes generally have a chromogenic marker and can be observed by traditional light microscopy. FISH probes generally have a fluorescent marker bonded and must be visualized with the use of a fluorescent microscope.

In one aspect, for in situ hybridization of paraffin-embedded tissues, sections of paraffin-embedded tissue immobilized on glass substrates are treated as follows: substrates are dewaxed in staining dishes by three changes in xylene for 2 minutes each (dewaxing is not necessary for non-embedded single cells); dewaxed samples are then rehydrated using the following procedure: exposure to 100% ethanol, two times for two minutes, then subsequent 2 minute incubations in 95%, 70%, and 50% ethanol. (It should be apparent to those of ordinary skill in the art that the incubation time is not critical and may be optimized, but in general should be at least two minutes.)

Samples are denatured (e.g., by incubation for 20 minutes at room temperature in 0.2 N HCl, followed by heat denaturation for 15 minutes at 70° C. in 2× SSC). Samples are then rinsed, for example, in 1× PBS for 2 minutes. In some situations, usually empirically determined, a pronase digestion step may be included here which later allows improved access of the probes to the nucleic acids contained within the tissue sections. In such cases, samples are digested for 15 minutes at 37° C. with pre-digested, lyophilized pronase at an empirically determined concentration which allows hybridization yet preserves the cellular morphology (e.g., such as 0.1 to 10 μg/ml).

Pronase-digested samples are incubated for 30 seconds in a wash buffer, such as 2 mg/ml glycine in 1×PBS, to stop the digestion process. Samples may be post-fixed, for example, using freshly prepared 4% paraformaldehyde in 1× PBS, for 5 minutes at room temperature. Fixation is stopped by further washes, e.g., a 5 minute incubation in 3× PBS, followed by two 30 second rinses in 1× PBS. Samples are then soaked in 10 mM DTT, 1× PBS, for 10 minutes at 45° C., followed by a 2 minute incubation in 0.1 M triethanolamine, pH 8.0 (triethanolamine buffer). Next, samples are placed in fresh triethanolamine buffer to which acetic anhydride is added to 0.25% final concentration, followed by mixing and 5 minutes' incubation with gentle agitation. In one aspect, more acetic anhydride is added to a final concentration of 0.5%, followed by 5 minutes' further incubation. Samples are washed, for example, for 5 minutes in 2× SSC, and by dehydrated by successive incubation in 50%, 70%, 95% and 100% ethanol for 2 minutes each at room temperature. Preferably, samples are air-dried or dried with desiccant before proceeding to the hybridization step. Any, or all, of the preceding series of steps may be automated in order to increase throughput.

Probes for in situ hybridization may be DNA or RNA oligonucleotides (e.g., RNA transcribed in vitro). In one aspect, RNA probes labeled with ³⁵S are dissolved in 50 mM dithiothreitol (DTT) and are added to a non-specific competitor. In one aspect, the competitor is preferably RNA made in the same manner as the labeled specific probe, except from a transcription template with non-specific sequences, such as a vector with no insert. No labeled ribonucleosides are in the reaction mix.

The probe/non-specific competitor mixture is then denatured, for example, by heating at 100° C. for 3 minutes, and added to a hybridization buffer (e.g., such as 50% (v/v) deionized formamide, 0.3 M NaCl, 10 mM Tris (pH 8.0), 1 mM EDTA, 1× Denhardt's solution, 500 mg/ml yeast tRNA, 500 mg/ml poly(A), 50 mM DTT, and 10% polyethylene glycol 6000) to a 0.3 μg/ml-10 μg/ml final probe concentration. An estimate of the amount of probe synthesized is based on a calculation of the percent of the label incorporated and the proportion of the labeled base in the probe molecule as a whole. In one aspect, the non-specific competitor is provided in an amount approximately equal to one half the mass of labeled probe.

The probe/hybridization mix is incubated at 45° C. until applied to the microarrays and test tissue sample as a thin layer of liquid. Hybridization reactions are generally incubated in a moist chamber such as a closed container containing towels moistened with 50% deionized formamide, 0.3 M NaCl, 10 mM Tris (pH 8.0), 1 mM EDTA, at 45° C. If background (e.g., the amount of non-specific labeling) proves to be a problem, a 1 to 2 hour pre-hybridization step using only non-specific, unlabeled riboprobe competitor in hybridization buffer can be added prior to the step in which labeled probe is applied.

In one aspect, hybridization is carried out for 30 minutes to 4 hours, followed by washing to remove any unbound probe. In one aspect, the profile array substrates are washed in an excess (100 ml each wash) of the following buffers: 50% formamide, 2× SSC, 20 mM β-mercaptoethanol, two times, for 15 minutes at 55° C.; 50% formamide, 2× SSC, 20 mM β-mercaptoethanol, 0.5% Triton X-100, two times, for 15 minutes at 55° C.; and 2× SSC, 20 mM β-mercaptoethanol, two times, for 2 minutes at 50° C.

In another aspect, samples are subjected to RNAse digestion for 15 minutes at room temperature for example using a solution containing 40 mg/ml RNase A, 2 mg/ml RNase T1, 10 mM Tris (pH 7.5), 5 mM EDTA and 0.3 M NaCl. In one ebmodiment, after RNase digestion, slides are soaked two times for 30 minutes each in 2× SSC, 20 mM β-mercaptoethanol at 50° C., followed by two washes in 50% formamide, 2× SSC, 20 mM β-mercaptoethanol at 50° C. and two washes of 5 minutes each in 2× SSC at room temperature. Hybridized, washed slides are dehydrated through successive two minute incubations in the following: 50% ethanol, 0.3 M ammonium acetate; 70% ethanol, 0.3 M ammonium acetate; 95% ethanol, 0.3 M ammonium acetate; 100% ethanol. Slides are air dried overnight and with emulsion for autoradiography according to standard methods.

Sections prepared from frozen tissues may be hybridized by a similar method except that the dewaxing and paraformaldehyde fixation steps are omitted. For details, see Ausubel et al., 1992, Short Protocols in Molecular Biology, (John Wiley and Sons, Inc.), pp. 14-15 to 14-16, the entirety of which is incorporated by reference herein. In still another aspect, ISH or FISH is performed with one or more amplification steps, i.e., such as by performing in situ PCR. A detailed description of these techniques are presented in Ausubel, et al., 1992, supra, pp. 14-37 to 14-49, the contents of which are hereby incorporated by reference.

In a further aspect of the invention, information obtained from a single sublocation on a microarray can be information relative to the expression of both proteins and nucleic acids. For example, in one aspect of the invention, after performing immunohistochemistry on tissue at a sublocation, a portion of the tissue is obtained to isolate nucleic acids which are further analyzed by amplification methods such as PCR. Detection of nucleic acids isolated from an embedded tissue sample is known in the art and is described in, for example, U.S. Pat. No. 6,013,461, U.S. Pat. No. 6,110,902, and U.S. Pat. No. 6,114,110, the entireties of which are incorporated by reference herein.

In still a further aspect, tissues can be counterstained to highlight their morphology (e.g., with hematoxylin/eosin, or another dye or combination of dyes, such as described in Ausubel et al., 1992, supra, pp. 14-19 to 14-22).

As with the IHC techniques described above, nucleic acid hybridization techniques can also be automated. In one aspect, both detection and probing is automated. For example, in one aspect, a profile array substrate which has been, or is being reacted, with a molecular probe is in communication with a detector. A light source in proximity to the tissue samples on the substrate transmits light to the samples and light transmitted by the samples is received by the detector. In one aspect, the detector is in communication with the tissue information system described above and signals transmitted to the tissue information system relating to optical information from the tissues are displayed and/or stored within the electronic database. In one aspect, optical information from tissue samples on the microarray is displayed as an image of tissue(s) on the interface of the display of a user device included in the tissue information system.

Kits

The invention further provides kits. A kit according to the invention, minimally contains a tissue microarray 13 and provides access to an information database (e.g., in the form of a URL and an identifier which identifies the particular microarray being used, and/or a password). In one aspect, the kit comprises instructions for accessing the database 5, or one or more molecular probes, for obtaining molecular profiling data using the microarray 13, and/or other reagents necessary for performing molecular profiling (e.g., labels, suitable buffers, and the like). In a preferred aspect, kits are provided which include a panel of molecular probes reactive with a plurality of GPCR pathway biomolecules.

### EXAMPLES

The invention will now be further illustrated with reference to the following examples. It will be appreciated that what follows is by way of example only and that modifications to detail may be made while still falling within the scope of the invention.

### Example 1

In one aspect, tissue microarrays 13, and preferably whole body microarrays 13, from a population of patients are reacted with one or more molecular probes for a GPCR, its ligand (e.g., a G protein), and cAMP. In some aspects, molecular probes reactive with one or more of phospholipase C, adenyl cyclase, phosphodiesterase, a GRK, protein kinase A, protein kinase C, adenosine kinase, receptor tyrosine kinases (RTK), a cytoplasmic tyrosine kinase, or other kinase, a protein-tyrosine phosphatase (PTP), a neurotransmitter, a neuropeptide, an RGS protein, a GAP protein, protein kinase C, integrin, paxillin, an extracellular matrix protein, p130(Cas), one or more ion channel proteins, thrombin, rho A, phosducin, phosducin-like protein (PhLP), an Erk protein, one or more Ca⁺⁺ dependent proteins, a hormone such as parathyroid hormone (PTH), lysophosphatidic acid (LPA), sphingosine-1-phosphate (S1P or SPP), are alternatively, or additionally, reacted with the tissue microarray 13.

The reactivity of different tissues on the microarrays 13 is determined and information related to reactivity is stored in a database 5 which includes patient information and/or other molecular profiling data relating to the tissues on the microarrays. In one aspect, tissue samples on the microarrays 13 are obtained from one or more of: normal patients, patients with bacterial, fungal, protozoan and viral infections, particularly infections caused by HIV-1 or HIV-2; patients with cancer; diabetes, obesity, anorexia, bulimia, asthma, Parkinson's disease, acute heart failure, hypotension, hypertension, urinary retention, osteoporosis, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette's syndrome, and other diseases or conditions. Molecular profiling information obtained from the tissue microarrays 13 is stored in the specimen-linked database 5.

In another aspect, the same microarrays 13 which have been evaluated to determine the expression of known pathway biomolecules are also evaluated to determine the expression of one or more unknown pathway biomolecules. For example, in one aspect, the arrays are probed with molecular probes (complementary sequences of, or predicted peptide products) of EST or cDNA sequences known to be expressed in tissues in which physiological responses to stroke have been observed (e.g., such as neural tissue). Information relating to the reactivity of the molecular probes with the various samples in the microarrays 13 is collected and inputted into the system 1 to be stored in expressed sequences subdatabases. The IMS 7 is then used to correlate and model the likely relationships of gene products represented by these ESTs or cDNAs with other molecules which have been identified as part of the molecular pathway(s) associated with the pathology of stroke, including gene products described in Koistinato et al., NeuroReport 8(2): i-iv (1997) such as the immediate early genes associated with ischemia (e.g., transcription factors in the Fos family such as c-fos, fos-B, Fra-1, Fra-2; transcription factors in the Jun family such as C-jun, junB, junD; transcription factors in the ATF/CREB family; transcription factors with Cys2-His2 zinc finger DNA-binding domains such as krox-24, zif268, egr1, NGFI-A, NGFI-B, NGFI-C, egr-2, egr-3, and Nurr1; and the 9-cis retinoic acid receptor), apoptosis genes (e.g., bax, bcl-x₅, BAD, ICE, or other accelerators of cell death; inhibitors of cell death such as bcl-2, bcl-x_{L}; p53); heat shock proteins, adhesion molecules (e.g., ICAM 1, P-selectin) and cytokines (e.g., TGF-β1, TNF-α, IL-1), nitric oxide synthase isoforms (nNOS, iNOS, eNOS), growth factors (NGF, BDNF, NT-3, bFGF, trkB) and their receptors.

In a preferred aspect, the IMS 7 simulates predicted pathways which include these gene products and ranks the likelihood that these pathways exist.

In a further aspect, gene products identified as part of a likely pathway are identified a drug targets to be used in screening assays to identify agents which can interact with these gene products. The physiological response of cells (e.g., in cell culture or in animal models) to these agents can be monitored by evaluating the effects of these agents on the expression of one or more pathway biomolecules in cell and/or tissue samples arrayed on additional microarrays 13.

### Example 2

In one aspect, a plurality of whole body tissue microarrays 13 is generated using tissue samples from an autopsy repository to create a database of information relating to the physiological responses of patients who have experienced one or more strokes. Preferably, the microarrays represent a population of patients who have either died from a stroke or who have died from other causes but who have had at least one stroke at some point in their lives.

In one aspect, the physiological responses of these patients to one or more strokes is evaluated by reacting whole body microarrays 13 derived from these patients with a plurality of probes which detect known pathway molecules whose expression has been correlated with the pathology of stroke (see, e.g., as described in Choi, Neuron 1: 623-634 (1988); Choi, Cereb. Brain Met. Rev. 2: 105-147; and Appel, Trends Neurosci. 16: 3-5 (1993); the entireties of which are incorporated herein). For example, in one aspect, one or more microarrays 13 are reacted with probes which specifically react with the products of glutamate receptor genes (including NMDA receptors; non-NMDA receptors, such as kainate receptors and AMPA/quisqualate receptors; and metabotropic receptors). Preferably, microarrays 13 are reacted with probes to detect the expression of both RNA and protein products of these genes. Identical sets of microarrays 13 (e.g., sections from the same recipient block, and preferably, sections within 100 μm of each other) are also reacted with molecular probes to glutamate itself and/or other substrates of these receptors (e.g., by providing labeled aptamer probes which specifically bind to these molecules). Additional molecular probes are used to assess the expression of ion channels dependent on the activation of these receptors and the expression of second messenger molecules whose levels are related to rises in levels of intracellular ions (e.g., such as Ca⁺⁺, Na⁺, and K⁺), such as is observed when these ion channels are activated. In a preferred aspect, molecular probes also are used to monitor the expression of Ca⁺⁺ dependent proteases, lipases, and endonucleases, and to monitor lipid peroxidation and other signs of cellular destruction associated with responses to stroke. In some aspects, an evaluation of the tissue microarrays also includes an evaluation of the morphology of individual tissue samples on the various microarrays 13 (e.g., by direct viewing or by obtaining images or other optical information from these samples).

Data from these analyses are inputted into the specimen-linked database 5 of the system 1 to create a “Response to Stroke” subdatabase. Because each microarray used is identified with an identifier which links the microarray to information already in the database relating to the characteristics of the patients who were the sources of the tissues on the array (e.g., such as the patient's medical histories), the IMS 7 can implement its relationship determining function to identify correlations between molecular expression patterns observed with these patient characteristics. Patient characteristics can include variables such as age at the time of death, sex, age at the time of the first stroke, number of strokes, length of time the patient was on particular medications, and the like. The IMS 7 can also compare records in the “Response to Stroke Database” to records in a “Normal Patient” database to identify which responses are most likely part of the pathology of stroke.

The Response to Stroke subdatabase is also further organized according to concurrent or underlying conditions (e.g., such as other diseases) to which a patient has been exposed. The database can therefore be compared to records in databases comprising information relating the specific concurrent or underlying conditions. For example, data in the Response to Stroke subdatabase from patients who had diabetes at the time of death could be compared to a subdatabase of information relating to tissue samples from patients with diabetes to identify common attributes in both subdatabases and to further define particular types of responses likely to be unique to stroke or diabetes respectively. In preferred aspects, the system 1 displays on a display of a user device 3, a comparison of expression profiles of pathway biomolecules in the various subdatabases.

### Example 3

In one aspect, a plurality of GPCRs for which no ligand is known are arrayed on a substrate to form a protein array. The GPCR protein array is contacted with any of: tissue samples, protein display libraries, protein fractions from tissues, and the like, to identify potential ligands of these GPCRs. Ligands which bind to the GPCRs or molecular probes which specifically react with these ligands are subsequently contacted with one or more whole body tissue microarrays 13 to determine the expression of the ligands in one or more tissues from normal and/or diseased patients. In a preferred aspect, information relating to the expression of putative ligands is stored in a specimen-linked database 5 and a tissue information system 1 as described above is used to model the likelihood that the ligand is involved in a particular GPCR pathway.

### Example 4

In one aspect, cells which have been modified to express a GPCR and isogenic cells which do not express the GPCR are arrayed on a microarray 13. The expression of one or more GPCR pathway biomolecules in both of these cell types is evaluated by reacting the microarray with probes specific for one or more GPCR pathway molecules. In a preferred aspect, the cells which express or do not express the GPCR are exposed to varying levels of agonists or antagonists for varying amount of times and the treated cells are also arrayed on the microarray. Preferably, information relating to the physiological responses of cells to the agonists and antagonists (e.g., rates of growth and death in exposed cells) is stored in a specimen-linked database 5 along with molecular profiling data obtained using the microarrays. The tissue information system 1 is used to correlate the physiological responses observed in vitro with the expression of GPCR pathway molecules.

### Example 5

In one aspect, tissue microarrays 13 are used to evaluate G protein uncoupling of GPCRs in response to a condition. For example, colocalization of GPCR and arrestin can be monitored by evaluating the colocalization of antibodies specific for each respective biomolecule. In preferred aspects, the tissue microarray 13 is contacted with one or more antibodies specific for protein kinases associated with the desensitization of a G protein coupled response, such as c-Jun amino-terminal kinase 3 (JNK3), apoptosis signal-regulating kinase 1 (ASK1), and mitogen-activated protein kinase (MAPK) kinase 4. Preferably, the tissue microarray 13 is contacted with antibodies which can distinguish the phosphorylated from the unphosphorylated form of a GPCR.

### Example 6

In one aspect, tissue microarrays from patients showing symptoms of a pathological immune response and tissue microarrays 13 from normal patients are reacted with molecular probes specific for one or more of G protein-coupled receptor kinases GRK1-6, (preferably GRK2, GRK3, and GRK6), GPCR substrates of these kinases, chemokines and PGs. The expression of these molecules can be correlated with the characteristics of patients who provided tissues for the arrays using the IMS 7. In a preferred aspect, the microarray 13 is a whole body tissue microarray 13 and is probed with differentially labeled probes which are specific for the phosphorylated and unphosphorylated forms of the GPCR (e.g., the uncoupled and coupled forms of the proteins), respectively. The microarrays 13 also preferably comprise at least one synovial fluid tissue sample and at least one microarray 13 is from a patient with arthritis.

Additionally or alternatively, the microarrays 13 can be reacted with one or more molecular probes specific for Th2 cells, Th1 cells, monocyte chemoattractant protein-3 (MCP-3), MCP-4 an eotaxin, an eosinophil-specific marker, integrin, CCR3, a chemokine, thrombin, histamine, Elk-1, activator protein-1, cyclin D1 expression, EGF, p42/p44, p70, a Cysteinyl leukotriene (CysLT), GPCR CysLT(1) and/or CysLT(2), leukotriene C(4) (LTC(4)) and leukotriene D(4) (LTD(4)). Preferably, the one or more probes are capable of distinguishing between phosphorylated and unphosphorylated forms of phosphorylated proteins. The microarrays also preferably comprise at least one lung tissue sample and at least one microarray is from a patient with asthma.

In still another aspect, tissues or cells are obtained from patients with sepsis and arrayed on microarrays. The microarrays can be reacted with molecular probes for one or more of an RGS 1, RGS 16, a GPCR, a vasocative GPCR agonist (e.g., angiotensin II, endothelin-1, alpha-thrombin), c-fos, activin, and other GPCR pathway biomolecules.

In one aspect, tissues are isolated from patients with an inflammatory disease and normal patients and used to generate microarrays. The microarrays can be reacted with molecular probes reactive with one or more of: a neuropeptide, including a bombesin-like peptide, nonreceptor tyrosine kinase p125fak, adaptor proteins (e.g., such as p130cas and paxillin), Rho, a CCR chemokine, and one or more molecules of a cell cycle pathway. Preferably, probes are used which are capable of distinguishing between the phosphorylated and unphosphorylated forms of one or more of these biomolecules.

In another aspect, the microarrays are probed with molecular probes (complementary sequences of, or predicted peptide products) of EST sequences or other expressed sequences to identify additional GPCR pathway molecules. Information relating to the reactivity of the molecular probes with the various samples in the microarrays is collected and inputted into the system 1 to be stored in an expressed sequence subdatabase. The IMS 7 is then used to correlate and model the likely relationships of gene products represented by these ESTs with other molecules which have been identified as part of molecular pathway(s) associated with the pathological immune responses. In a preferred aspect, the IMS 7 simulates predicted pathways which include these gene products and ranks the likelihood that these pathways exist.

In a further aspect, gene products identified as part of a likely pathway are identified a drug targets to be used in screening assays to identify agents which can interact with these gene products. The physiological response of cells (e.g., in cell culture or in animal models) to these agents can be monitored by evaluating the effects of these agents on the expression of one or more pathway biomolecules in cell and/or tissue samples arrayed on additional microarrays.

### Example 7

In one aspect, tissue microarrays from a population of patients infected with the HIV virus are contacted with molecular probes specific for Kaposi's sarcoma-associated herpesvirus G protein-coupled receptors (KSHV-GPCRs) and one or more human GPCR pathway molecules (e.g., such as thyrotropin-releasing hormone (TRH) receptors or ml-muscarinic-cholinergic receptors, ion channels, and Ca⁺⁺ dependent proteins) to evaluate the G protein uncoupling of these molecules (see, e.g., Lupu-Meiri et al., J. Biol. Chem. (2000), the entirety of which is incorporated by reference herein). In some aspects, the microarrays are additionally reacted with molecular probes specific for one or more of c-jun amino terminal kinase/stress-activated protein kinase, lin kinase, and proline-rich tyrosine kinase 2. p38 mitogen-activated protein kinase, and (IFN)-gamma-inducible protein 10 (HuIP-10). Preferably, antibodies which distinguish between the phosphorylated and non-phosphorylated forms of pathway proteins are used as probes. Molecular profiling information obtained from these assays is stored within the specimen-linked database 5 and the IMS 7 is used to correlate this information with patient characteristics, such as drugs being used, stage of the disease, and the like.

### Example 8

In one aspect, microarrays from a plurality of patients are reacted with allele-specific molecular probes capable of recognizing each of 19 published missense mutations in the OA1 GPCR and wild type OA1 (see e.g., d'Addio et al., Hum. Mol. Genet. 9(20):3011-8 (2000). In a preferred aspect, the reaction of probes recognizing mutant OA1 GPCRs is correlated with the presence or absence of ocular albinism, reductions in visual acuity, hypopigmentation of the retina, and the presence of macromelanosomes in the skin and eyes, and other patient characteristics. Patient samples can additionally be genotyped to determine heterozygosity or homozygosity for mutant alleles in parallel with microarray analysis. Molecular profiling information obtained from these assays is stored within the specimen-linked database 5 and the IMS 7 is used to correlate this information with patient characteristics and other clinical information.

### Example 9

In one aspect, tissue microarrays from a population of patients are contacted with one or more molecular probes which specifically react with a somatostatin (e.g., somatostatin 14 and/or 28), and/or pituitary Growth Hormone, and/or a somatostatin receptor. In a preferred aspect, the expression of the one or more molecules is correlated with patient characteristics, including the presence or absence of a neuropsychiatric disorder or other behavioral disorder. Molecular profiling information obtained from these assays is stored within the specimen-linked database 5 and the IMS 7 is used to correlate this information with patient characteristics and other clinical information.

### Example 10

In one aspect, a plurality of tissue microarrays are obtained from a population of patients having a sleep disorder are contacted with molecular probes specific for a GPCR, and a member of the fos family of immediate early genes (IEGs). In a preferred aspect, an EST or cDNA library or array is probed with labeled nucleic acids from a patient having a sleep disorder, while another EST or cDNA library or array is probed with labeled nucleic acids from a normal patient. ESTs/cDNAs which are differentially expressed in patients having the sleep disorder are used to generate molecular probes (e.g., complementary nucleic acid sequences, or antibodies reactive with expressed peptides) which are contacted with whole body tissue microarrays, preferably from the same patient used to generate the EST/cDNA library or array. An identification of a differentially expressed sequence in a tissue microarray is used to validate results obtained in the library or array and validated expressed sequences are used to express peptides which are identified as candidate drug targets for use in screening to identify lead drug compounds for the treatment of the sleep disorder.

### Example 11

In one aspect, tissue microarrays from a plurality of patients having a pathology associated with abnormal bone growth and from normal patients are reacted with molecular probes specific for one or more of a GPCR, a GPCR kinase such as GRK2, a beta-arrestin, a MAP kinase, insulin-like growth factor-1 (IGF-1), a BMP, and an OCIF to obtain molecular profile data from these patients which can be linked to information regarding patient characteristics using the specimen-linked database 5. Preferably, molecular probes capable of distinguishing between the phosphorylated and unphosphorylated forms of the GPCR(s) are used.

### Example 12

In one aspect, tissue microarrays from a plurality of patients having a pathology associated with neointimal hyperplasia and from normal patients are reacted with molecular probes specific for one or more of a GPCR, GPCR kinase-2, endothelin-1, angiotensin II, thrombin, thromboxane A(2), PDGF, PDGF-beta receptor, EGF, EGFr, epidermal growth factor receptor, and preferably, one or more cell cycle biomolecules, to obtain molecular profile data from these patients which can be linked to information regarding patient characteristics using the specimen-linked database 5.

### Example 13

In one aspect, in vitro cell culture assays are performed in which the expression, function, and ligand-dependent trafficking of GPCR-green fluorescent protein (GFP) fusion conjugates stably transfected into cells (e.g., such as HEK 293 cells) are determined and correlated with the expression of one or more GPCR pathway molecules in tissue microarrays derived from the cells being assayed. For example, transfected cells exposed to varying levels of peptide mimetics, agonists or antagonists, for varying amounts of time in a plurality of assays are evaluated to determine parameters such as the amount of GPCR expressed, binding kinetics between the GPCR and its ligand, and the growth rate and death rate of cells. Information relating to these parameters is stored in the specimen-linked database 5. A sample of cells from each of the assays is obtained and embedded to provide donor blocks for generating recipient blocks representing cells from the plurality of assays. Microarrays obtained from the recipient blocks are then contacted with a GPCR pathway probe, and preferably, with a plurality of GPCR pathway probes, and information relating to the reactivity of the probes with individual samples on the microarrays is correlated with the physiological responses of the samples in vitro using the IMS 7.

### Example 14

In one aspect, tissue microarrays are obtained from a population of patients with heart disease and from a population of normal patients and reacted with a molecular probe specific for one or more of a GPCR, GRK2, GRK3, endothelin, phenylephrine, MAPK cascade biomolecules, including, but not limited to, any one or more of biomolecules of the extracellularly regulated kinase cascade, the stress-activated protein kinase/c-Jun N-terminal kinase cascade, the p38 MAPK cascade, and the protein kinase B pathway. Molecular profiling information obtained from these assays is stored within the specimen-linked database 5 and the IMS 7 is used to correlate this information with patient characteristics and other clinical information.

### Example 15

In one aspect, tissue microarrays are obtained from a population of patients and reacted with a molecular probe specific for one or more of prolactin, a GPCR, cAMP, cytochrome P450, and one or more molecules in the follicle-stimulating hormone receptor (FSHR) transduction pathways, gonadotrophin-releasing hormone receptor pathway (GnRH), and luteinizing hormone/human chorionic gonadotrophin (LH/HCG) pathway. Preferably, probes which are specific for glycosylated and non glycosylated forms of proteins in these pathways are reacted with the microarrays. Molecular profiling information obtained from these assays is stored within the specimen-linked database 5 and the IMS 7 is used to correlate this information with patient characteristics and other clinical information.

### Example 16

In one aspect, tissue microarrays are generated from a population of patients having eating disorders and normal patients and the expression of one or more of melanin-concentrating hormone receptor, melanin concentrating hormone, the GPCR SLC-1, a G(alpha)i and/or G(alpha)q protein is determined. Information relating to this expression is stored in the specimen-linked database 5 and correlated with data relating to the expression of other genes to identify candidate molecules which belong to the SLC-1 pathway using the IMS 7.

All literature citations, patents, and patent publications cited herein are incorporated by reference in their entirety. Variations, modifications, and other implementations of what is described herein will occur to those of ordinary skill in the art without departing from the spirit and scope of the invention as claimed. Accordingly, the invention is to be defined not by the preceding illustrative description but instead by the spirit and scope of the following claims.

What is claimed is:

1. An information system comprising:
a tissue microarray having a plurality of samples;
a specimen-linked database containing a plurality of clinical information of each sample and a plurality of information relating to G-protein coupled receptor pathway molecules of each sample; and
an information management system for searching the specimen-linked database and determining relationships between the plurality of clinical information and the plurality of information relating to G-protein coupled receptors pathway biomolecules.

2. The information system of claim 1 further comprising a plurality of tissue microarrays.

3. The information system of claim 2 wherein an at least one tissue microarray is a control tissue microarray.

4. The information system of claim 1 wherein the tissue microarray comprises a plurality of sublocations.

5. The information system of claim 4 wherein each of the plurality of sublocations comprises a set of coordinates.

6. The information system of claim 4 wherein each of the plurality of sublocations comprises a tissue sample wherein the tissue sample has an at least one known biological characteristic.

7. The information system of claim 1 wherein the tissue microarray comprises a substrate for handling of the tissue microarray through at least one molecular procedure.

8. The information system of claim 7 wherein the substrate comprises a location for placing an identifier.

9. The information system of claim 1 wherein the plurality of samples are a plurality of tissues.

10. The information system of claim 1 wherein the plurality of samples are a plurality of cells.

11. The information system of claim 1 wherein each of the plurality of samples are derived from a single organism.

12. The information system of claim 1 wherein each of the plurality of samples has a similar trait.

13. The information system of claim 1 wherein each of the plurality of samples comprise a particular patient demographic group.

14. The information system of claim 1 wherein each of the plurality of samples are taken from a plurality of individuals with a particular disease.

15. The information system of claim 14 wherein the disease is a neurodegenerative disease.

16. The information system of claim 14 wherein the disease is a neuropsychiatric disease.

17. The information system of claim 1 wherein the plurality of samples represents a plurality of different stages of a cell proliferative disorder.

18. The information system of claim 1 wherein each of the plurality of samples are taken from a plurality of individuals who have been exposed to a drug.

19. The information system of claim 1 wherein each of the plurality of samples is taken from a plurality of individuals who have been exposed to an environmental condition.

20. An information system comprising:
a tissue microarray having a plurality of samples;
a specimen-linked database containing a plurality of clinical information of each sample and a plurality of information relating to G-protein coupled receptor pathway molecules of each sample;
an information management system for searching the specimen-linked database and determining relationships between the plurality of clinical information and the plurality of information relating to G-protein coupled receptors pathway biomolecules; and
a user device connected to a network.

21. The information system of claim 20 further comprising a plurity of user devices located at a plurality of physical locations.

22. The information system of claim 20 wherein the user device comprises an interface.

23. The information system of claim 22 wherein the interface provides a link to an identifier associated with the tissue microarray.

24. The information system of claim 23 wherein the interface further provides a link to a set of coordinates wherein the set of coordinates represents a single sample of the plurality of samples.

25. The information system of claim 22 wherein the interface comprises a link to the specimen-linked database.

26. The information system of claim 20 wherein the user device comprises a processor.

27. The information system of claim 20 wherein the user device comprises an operating system.

28. The information system of claim 27 wherein the user device further comprises a web browser.

29. The information system of claim 20 wherein the user device comprises a text input element.

30. The information system of claim 20 wherein the user device is coupled to a navigating device.

31. The information system of claim 20 further comprising an at least one server.

32. The information system of claim 31 wherein the at least one server provides access to an at least one data storage media.

33. The information system of claim 20 wherein the information management system further comprises an application program wherein the application program is capable of searching the specimen-linked database.

34. The information system of claim 20 further comprising an information output module wherein the information output module is capable of outputting and reporting information from the specimen-linked database.

35. The information system of claim 20 further comprising an information input module.

36. The information system of claim 20 wherein the user device is coupled to a molecular profiling system.

37. An information system comprising:
at least one tissue microarray wherein the at least one tissue micro array includes a plurality of sublocations wherein each sublocation comprises a sample;
a specimen-linked database containing a plurality of clinical information of each sample and a plurality of information relating to G-protein coupled receptor pathway molecules of each sample; and
an information management system for searching the specimen-linked database and determining relationships between the plurality of clinical information and the plurality of information relating to G-protein coupled receptors pathway biomolecules.

38. The information system of claim 37 wherein the specimen-linked database includes a data model wherein the data model organizes a plurality of information.

39. The information system of claim 38 wherein the data model is selected from the group consisting of a flat file model, an indexed file model, a network data model, a hierarchial data model, and a relational data model.

40. The information system of claim 37 wherein the specimen-linked database comprises a database dictionary having a set of parameters specified by a system operator.

41. The information system of claim 40 wherein the database dictionary further comprises a plurality of word equivalents for searching the specimen-linked database.

42. The information system of claim 41 wherein the database dictionary further comprises a plurality of codes for searching the specimen-linked database.

43. The information system of claim 42 wherein the plurality of codes are selected from the group consisting of a plurality of SNOWMED codes, a plurality of DSM-IV-TR codes, and a plurality of UNIGENE codes.

44. The information system of claim 37 further comprising a temporary database wherein a plurality of information entered into the temporary database is subject to validation by a system operator prior to the plurality of information being included into the specimen-linked database.

45. The information system of claim 37 wherein the specimen-linked database comprises a plurality of information representing a whole body tissue microarray.

46. The information system of claim 37 wherein the specimen-linked database comprises information from a plurality of tissue microarrays.

47. The information system of claim 37 wherein the specimen-linked database comprises information relating to a plurality of samples from a plurality of different recombinant inbred strains of a single individual.

48. The information system of claim 37 wherein the specimen-linked database comprises a plurality of information obtained from an at least one sample assayed independently of the tissue miocroarray.

49. The information system of claim 37 wherein the specimen-linked database comprises a plurality of subdatabases wherein each subdatabase comprises information relating to a particular category of sample information.

50. The information system of claim 37 wherein the specimen-linked database comprises a series of uncompressed raw data files.

51. The information system of claim 37 wherein the specimen-linked database further comprises a genomic medicine database wherein the genomic medicine database comprises a plurality of subdatabases comprising a set of information about a plurality of GPCR pathway biomolecules.

52. The information system of claim 37 wherein the specimen-linked database further comprises a physiological response database comprising information relating to a plurality of physiological responses of patients to a plurality of conditions.

53. The information system of claim 52 wherein the physiological response database comprises a plurality of information relating to kinetic parameters which govern a plurality of physiological responses.

54. The information system of claim 52 wherein the physiological response database comprises a plurality of information relating to a plurality of biomolecules which are expressed or inhibited upon activation of a particular GPCR pathway biomolecule.

55. The information system of claim 52 wherein the physiological response database comprises a plurality of information relating the expression of at least one DNA repair gene.

56. The information system of claim 52 wherein the physiological response database comprises a plurality of information relating the expression of an at least one biomolecule involved in cholesterol metabolism.

57. The information system of claim 52 wherein the physiological response database comprises a plurality of information relating the expression of an at least one biomolecule involved in apoptosis.

58. The information system of claim 52 wherein the physiological response database comprises a plurality of information relating the expression of an at least one biomolecule involved in blood clotting.

59. The information system of claim 52 wherein the physiological response database comprises a plurality of information relating the expression of an at least one biomolecule involved in the flt-3 pathway.

60. The information system of claim 52 wherein the physiological response database comprises a plurality of information relating the expression of an at least one biomolecule involved in the JAK/STATS signaling pathway.

61. The information system of claim 52 wherein the physiological response database comprises a plurality of information relating the expression of an at least one biomolecule involved in a MAP kinase signaling pathway.

62. The information system of claim 52 wherein the physiological response database comprises a plurality of information relating the expression of an at least one biomolecule involved in a PI 3 kinase pathway.

63. The information system of claim 52 wherein the physiological response database comprises a plurality of information relating the expression of an at least one biomolecule involved in a ras activation pathway.

64. The information system of claim 52 wherein the physiological response database comprises a plurality of information relating the expression of an at least one biomolecule involved in a SIP signaling pathway.

65. The information system of claim 52 wherein the physiological response database comprises a plurality of information relating the expression of an at least one biomolecule involved in a TGF-β signaling pathway.

66. The information system of claim 52 wherein the physiological response database comprises a plurality of information relating the expression of an at least one biomolecule involved in a T cell receptor based signaling pathway.

67. The information system of claim 52 wherein the physiological response database comprises a plurality of information relating the expression of an at least one biomolecule involved in a MHC-1 mediated antigen presentation.

68. The information system of claim 52 wherein the physiological response database comprises a plurality of information relating to the expression of a plurality of pathway molecules in addition to GPCR pathway molecules expressed within whole body tissue microarrays obtained from a plurality of patients.

69. The information system of claim 68 wherein the physiological response database further comprise a plurality of subdatabases including information relating to a plurality of specific pathways.

70. The information system of claim 68 further comprising a plurality of information relating to the biological impact of the expression of the plurality of GPCR pathway biomolecules.

71. The information system of claim 52 wherein the physiological response database comprises a plurality of information relating to the expression of an at least one tyrosine kinase pathway molecule.

72. The system of claim 37 wherein the specimen-linked database comprises a plurality of information relating to a plurality of treatment options.

73. An information system comprising:
an at least one tissue microarray wherein the at least one tissue microarray comprises a plurality of sublocations wherein each sublocation comprises a sample;
a specimen-linked database containing a plurality of clinical information of each sample and a plurality of information relating to G-protein coupled receptor pathway molecules of each sample;
an information management system for searching the specimen-linked database and for determining relationships between the plurality of clinical information and the plurality of information relating to G-protein coupled receptors pathway biomolecules; and
a user device connected to a network.

74. The information system of claim 73 wherein the information management system displays a plurality of information on an interface of the user device.

75. The information system of claim 73 wherein the information management system is stored within an at least one server and the information management system is accessible remotely through the network.

76. The information system of claim 73 wherein the information management system is accessible through a readable medium.

77. The information system of claim 73 wherein the information management system is capable of understanding a set of natural language query terms.

78. The information system of claim 73 wherein the information management system is capable of understanding Boolean operators and truncation symbols.

79. The information system of claim 73 wherein the information management system generates a plurality of search data from a plurality of terms inputted into an interface of the user device and transfers the plurality of search data to an at least one search engine to initiate a search.

80. The information system of claim 73 wherein the information management system generates a plurality of search data through a selection of options which are displayed on an interface.

81. The information system of claim 73 wherein the information management system is capable of mapping a plurality of data points obtained from the specimen-linked database.

82. The information system of claim 73 wherein the information management system is cabable of classifying a plurality of tissue information by type or attribute.

83. The information system of claim 73 wherein the information management system is capable of assigning a relationship identification number to each of a plurality of attributes and storing the relationship identification numbers in the specimen-linked database where the attributes are indexed by the relationship identification number and provided with a descriptor.

84. The information system of claim 73 wherein the information management system comprises a statistical program to identify a plurality of attributes as representing a particular relationship.

85. The information system of claim 84 wherein the information management system is capable of analyzing a particular relationship between a plurality of data in the specimen-linked database using any of the methods in the group consisting of regression, decision trees, neural networks, and fuzzy logic.

86. The information system of claim 73 wherein the information management system further comprises an expert system.

87. The information system of claim 86 wherein the expert system is capable of identifying biomolecules which are likely to belong to a GPCR pathway.

88. The information system of claim 87 wherein the expert system further comprises a transaction manager wherein the transaction manager directs and outputs requests between an at least one server of the information system and an at least one interface of an at least one user devices of the system.

89. The information system of claim 88 further comprising an expression profile interface on which is displayed a representation of an at least one level of expression of an at least one selected GPCR pathway biomolecule in a plurality of samples.

90. The information system of claim 89 further comprising an at least one information category link identifying a plurality of physiological response categories.

91. A method of relating a plurality of information comprising:
creating a tissue microarray wherein the tissue microarray includes a plurality of sublocations and a plurality of samples wherein each sublocation includes a sample;
identifying the tissue microarray with an identifier and identifying each sublocation with a set of coordinates;
treating each of the plurality of samples with a molecular probe;
entering a plurality of clinical information relating to each sample into a specimen-linked database and entering a plurality of information relating to at least one G-protein coupled receptor pathway biomolecule of each sample into the specimen-linked database; and
correlating the identifier of the tissue microarray and the coordinates of a sublocation with the plurality of clinical information and the plurality of information relating to at least one G-protein coupled receptor pathway biomolecule of the sublocation using an information management system.

92. The method of claim 91 further comprising a plurality of tissue microarrays.

93. The method of claim 92 wherein an at least one tissue microarray is a control tissue micro array.

94. The method of claim 91 wherein each of the plurality of sublocations comprises a tissue sample wherein the tissue sample has an at least one known biological characteristic.

95. The method of claim 91 wherein the plurality of samples are a plurality of tissues.

96. The method of claim 91 wherein each of the plurality of samples are derived from a single organism.

97. The method of claim 91 wherein each of the plurality of samples has a similar trait.

98. The method of claim 91 wherein each of the plurality of samples comprise a particular patient demographic group.

99. The method of claim 91 wherein each of the plurality of samples are taken from a plurality of individuals with a particular disease.

100. The method of claim 91 wherein the plurality of samples represents a plurality of different stages of a cell proliferative disorder.

101. The method of claim 91 wherein at least one of the plurality of samples is taken from an individual that has been exposed to a drug.

102. The method of claim 91 wherein at least one of the plurality of samples is taken from an individual that has been exposed to an environmental condition.

103. The method of claim 91 further comprising displaying a link to the identifier associated with the tissue microarray wherein the link is displayed on an interface of a user device.

104. The method of claim 91 further comprising displaying a link to the set of coordinates wherein the set of coordinates represents a single sample of the plurality of samples.

105. The method of claim 91 further comprising displaying a link to the specimen-linked database.

106. The method of claim 91 wherein the interface allows a user to enter search terms.

107. The method of claim 91 further comprising searching the specimen-linked database with an information management system.

108. A method of relating a plurality of information comprising:
creating a tissue microarray wherein the tissue microarray includes a plurality of sublocations and a plurality of samples wherein each sublocation includes a sample;
entering a plurality of clinical information relating to each sample into a specimen-linked database and entering a plurality of information relating to at least one G-protein coupled receptor pathway biomolecule of each sample into the specimen-linked database;
correlating the identifier of the tissue microarray and the coordinates of a sublocation with the plurality of clinical information and the plurality of information relating to at least one G-protein coupled receptor pathway biomolecule of the sublocation using an information management system; and
searching the specimen-linked database with the information management system and displaying a plurality of search results on an interface of a user device.

109. The method of claim 108 further comprising comparing a first plurality of information, wherein the first plurality of information has been incorporated into the specimen-linked database, with a second plurality of information, wherein the second plurality of information has not been incorporated into the specimen-linked database.

110. The method of claim 109 wherein the information management system is capable of determining a plurality of relationships between the first plurality of information and the second plurality of information.

111. The method of claim 110 further comprising displaying the plurality of relationships on the interface of the user device.

112. The method of claim 108 wherein the user device is coupled to a molecular profiling system.

113. The method of claim 108 wherein the specimen-linked database includes a data model wherein the data model organizes a plurality of information.

114. The method of claim 108 wherein the information management system is stored within an at least one server and the information management system is accessible remotely through a network.

115. The method of claim 108 wherein the information management system is capable of understanding a set of natural language query terms.

116. The method of claim 108 wherein the information management system is capable of understanding Boolean operators and truncation symbols.

117. The method of claim 108 wherein the information management system generates a plurality of search data through a selection of options which are displayed on the interface.

118. The method of claim 108 wherein the information management system is capable of mapping a plurality of data points obtained from the specimen-linked database.

119. The method of claim 108 wherein the information management system is capable of classifying a plurality of tissue information by a type or an attribute.

120. The method of claim 108 wherein the information management system is capable of assigning a relationship identification number to each of a plurality of attributes and storing the relationship identification numbers in the specimen-linked database where the plurality of attributes are indexed by the relationship identification number and provided with a descriptor.

121. The method of claim 108 wherein the information management system comprises a statistical program to identify a plurality of attributes as representing a particular relationship.

122. The method of claim 121 wherein the information management system is capable of analyzing a particular relationship between a plurality of data in the specimen-linked database using any of the methods in the group consisting of regression, decision trees, neural networks, and fuzzy logic.

123. The method of claim 108 wherein the information management system is coupled to an expert system.

124. The method of claim 123 wherein the expert system is capable of identifying biomolecules which are likely to belong to a GPCR pathway.

125. The method of claim 124 wherein the expert system further comprises a transaction manager wherein the transaction manager directs and outputs requests between an at least one server and an at least one interface of an at least one user device of the system.

126. The method of claim 125 further comprising displaying a representation of an at least one level of expression of an at least one selected GPCR pathway biomolecule in a plurality of samples.
